(19) United States
(12) Patent Application Publication
(10) Pub. No.: US 2023/0279355 A1
(43) Pub. Date: Sep. 7, 2023

(54) METHOD FOR THE IN VITRO OR EX VIVO AMPLIFICATION OF STEM CELLS OF BROWN OR BEIGE ADIPOCYTES

(71) Applicants: CENTRE NATIONAL DE LA RECHERCHE SCIENTIFIQUE, Paris (FR); CHU DE NICE, Nice (FR); ETABLISSEMENT FRANCAIS DU SANG, Saint-Denis (FR); INSTITUT NATIONAL DE LA SANTE ET DE LA RECHERCHE MEDICALE, Paris (FR); UNIVERSITE COTE D'AZUR, Nice (FR); UNIVERSITE TOULOUSE III - PAUL SABATIER, Toulouse (FR)

(72) Inventors: Christian, Jean, Lucien Dani, Nice (FR); Vincent Dani, Nice (FR); Louis Casteilla, Toulouse (FR); Alain, Pierre, Louis Doglio, Saint André de la Roche (FR); Philippe Letertre, La Colle sur Loup (FR)

(73) Assignees: CENTRE NATIONAL DE LA RECHERCHE SCIENTIFIQUE, Paris (FR); CHU DE NICE, Nice (FR); ETABLISSEMENT FRANCAIS DU SANG, Saint-Denis (FR); INSTITUT NATIONAL DE LA SANTE ET DE LA RECHERCHE MEDICALE, Paris (FR); UNIVERSITE COTE D'AZUR, Nice (FR); UNIVERSITE TOULOUSE III - PAUL SABATIER, Toulouse (FR)

(21) Appl. No.: 18/016,128

(22) PCT Filed: Jul. 15, 2021
(86) PCT No.: PCT/EP2021/069888

§ 371 Date: Jan. 13, 2023

(30) Foreign Application Priority Data
Jul. 15, 2020 (FR) 2007407

(51) Int. Cl. C12N 5/077

(52) U.S. Cl. CPC C12N 5/0653; C12N 2502/1305; C12N 2502/1382; C12N 2502/28; C12N 2501/115; C12N 2501/105; C12N 2501/11; C12N 2503/02; C12N 2509/10

(57) ABSTRACT

The method for the in vitro or ex vivo amplification of stem cells of brown or beige adipocytes includes: extracting (i) a stromal vascular fraction from human adipose tissue including endothelial cells of the vascular network of human adipose tissue and stem cells of brown or beige human adipose tissue and (ii) an extracellular matrix of the human adipose tissue, the extracellular matrix including endothelial cells of the vascular network of human adipose tissue, stem cells of brown or beige human adipose tissue and collagen; mixing the stromal vascular fraction and the extracellular matrix; and culturing the mixture obtained, in suspension, in a culture medium.

### TECHNICAL FIELD OF THE INVENTION

The present invention relates to a method for the in vitro or ex vivo amplification of stem cells of brown or beige adipocytes from human adipose tissue. It further relates to a method for the in vitro or ex vivo amplification of stem cells of brown or beige adipocytes, an extracellular matrix, a composition comprising a mixture of an extracellular matrix and a stromal vascular fraction, a kit comprising this composition, a use of extracellular matrix or the composition comprising a mixture of the extracellular matrix and the stromal vascular fraction, stem cells of brown or beige adipocytes and brown or beige adipocytes, obtained according to the method of the invention for the use thereof.

### PRIOR ART

Cell therapy consists in a cell transplant aimed at restoring the functions of a tissue or organ when they are altered by an accident, a pathology, aging and metabolic disorders. It allows a long-term treatment of a patient thanks to an injection of cells called “therapeutic” cells. These cells are obtained, in particular, from multipotent stem cells from the patient himself.

Consideration was given to improving the metabolic flexibility and/or stimulating the energy dependence of a patient by increasing the mass of brown/beige adipose tissue (BAT) in this patient. This is an innovative approach intended to fight against metabolic diseases, such as diabetes, cardiovascular diseases and other metabolic dysfunctions. Indeed, brown or beige adipose tissue participates in the heat dissipation of the organism, in the control of the redox metabolism and it plays an endocrine and paracrine regulatory role through the hormone secretion. Restoring the function called “TAB” function in these patients therefore represents an attractive therapeutic option.

However, autologous cell therapies implementing brown or beige adipose tissue does not exist in practice, and this, for a main reason which is the virtual absence of a source of brown adipocytes in adult patients, more particularly in obese patients, for an in vitro or ex vivo amplification. Indeed, unlike white adipose tissue present in abundance and especially in obese patients, brown or beige adipose tissue is particularly rare in adult men and even almost non-existent in obese patients, for whom this virtual non-existence constitutes an aggravating factor, if this is not a major cause, of metabolic disorders in particular, but not exclusively, related to overweight.

In this context, there is a need to carry out cultures of autologous brown or beige adipocytes and, subsequently, develop cell amplification methods allowing obtaining significant amounts of therapeutic grade brown or beige adipocytes to fight metabolic diseases associated with obesity, such as diabetes, cardiovascular diseases and other metabolic dysfunctions.

The standard procedure for isolating and amplifying the adipocyte precursors from adipose tissue samples, goes through an enzymatic dissociation then through the expansion thereof in two dimensions (2D) by attachment to the plastic of culture dishes. This procedure is expensive, time-consuming, and requires numerous manipulations which increase the risk of contamination. In addition, it leads to a destruction of the three-dimensional structure of the tissue, as well as the loss of cell types of interest such as endothelial cells which play an essential role both for the vascularisation of the graft and the physiology of the adipocyte.

The non-enzymatic dissociation of adipose tissue appears to be an alternative method which is much cheaper, faster and which has undeniable advantages for the manufacture of a product which complies with the standards of a therapeutic grade production (reduced exposure to external products or contaminants). However, the non-enzymatic dissociation methods presented to date are not satisfactory because several studies report that the obtained number of adipocyte precursors is low compared to the enzymatic dissociation. In addition, the mature adipocytes, the extracellular matrix as well as the three-dimensional structure of adipose tissue is always lost at the end of the dissociation process, as well as the endothelial cells, after culture.

Different synthetic matrices have been proposed to seed the adipocyte precursors therein and thus try to reconstitute the structure of adipose tissue as well as possible. These matrices would also be used to in vitro orient the adipocyte precursors to a non-adipose cell type, essentially bony or cartilaginous, before implantation. Decellularised adipose tissue has also been proposed to increase the differentiation of the precursors and better mimic the structure of the adipose tissue. The manufacture of these types of matrices requires many steps involving enzymatic reactions or long chemical treatments. In addition, decellularised tissue, by definition, loses these endogenous cells. Adipose tissue which is undecellularised and enriched with adipocyte precursors (previously isolated by enzymatic dissociation) followed by a 2D amplification has recently been proposed as a matrix for a better bone reconstruction. The time to generate this biological matrix is long, requires three weeks of in vitro culture, and does not allow the amplification of the adipocyte precursors. Only interest in bone repair was highlighted by the authors.

The three-dimensional (3D) suspension culture represents an alternative method of choice to the 2D standard method because it essentially allows keeping the structure and the intrinsic qualities of the tissue. This advantage is significant because, for example, the absence of a relevant human model, which best in vitro mimics the adipose tissue, is a major limitation during preclinical phase testing, for the discovery of new effective drugs to fight obesity and associated metabolic diseases such as type 2 diabetes and cardiovascular diseases. In addition, the 3D culture is achievable in a closed system, which reduces the manipulations and the risks of contamination.

### SUMMARY OF THE INVENTION

In view of the above, a technical problem which the invention addresses is to in vitro or ex vivo obtain a large amount of stem cells in particular therapeutic grade brown or beige adipocytes from human white adipose tissue.

The solution of the invention to this technical problem has for first object, a method for the in vitro or ex vivo amplification of stem cells of brown or beige adipocytes, comprising the following steps: extracting, on the one hand, a stromal vascular fraction from human adipose tissue comprising endothelial cells of the vascular network of human adipose tissue and stem cells of human adipose tissue and, on the other hand, an extracellular matrix of said human adipose tissue, said extracellular matrix comprising endothelial cells of the vascular network of human adipose tissue, stem cells of human adipose tissue and collagen, the extraction of said extracellular matrix comprising a mechanical dissociation step; mixing said stromal vascular fraction and said extracellular matrix; and culturing the mixture obtained in the preceding step, in suspension, in a cell proliferation medium.

Thus, the suspension culture of the stromal vascular fraction, made possible thanks to the presence of the extracellular matrix, allows a 3D amplification, giving access to a large number of cells in an environment of the native adipose tissue and thus limiting the manipulations which increase the risk of contaminations.

Advantageously, —the mechanical dissociation step is a dissociation step which does not involve collagenase; —the mechanical dissociation step is a non-enzymatic dissociation step; —the extraction of the stromal vascular fraction and the extracellular matrix comprises the following steps: centrifugation of human adipose tissue to obtain at least two distinct fractions, a fraction A comprising a centrifuged extracellular matrix, and the stromal vascular fraction; and mechanical dissociation of the fraction A to obtain the extracellular matrix; —the method further includes a step of eliminating the blood cells present in the stromal vascular fraction; —the collagen of the extracellular matrix is structured collagen, which has a fibrillar organisation; —culturing the mixture of said stromal vascular fraction and said extracellular matrix comprises the following steps: transferring said mixture, in a sterile manner, into a suspension culture bag comprising the proliferation medium; amplifying said mixture to obtain an amplified mixture comprising cell clusters; —the amplified mixture comprising the cell clusters is the subject of a mechanical dissociation to obtain cell aggregates; —the proliferation medium comprises a serum, a fibroblast growth factor and an insulin-like growth factor; and —the method further includes a cell sorting step aimed at sorting the stem cells expressing the surface marker DPP4.

According to a second object, the invention relates to a method for in vitro or ex vivo obtaining brown or beige adipocytes comprising the following steps: extracting, on the one hand, a stromal vascular fraction from a human adipose tissue comprising endothelial cells of the vascular network of human adipose tissue and stem cells of human adipose tissue and, on the other hand, an extracellular matrix of said human adipose tissue, said extracellular matrix comprising endothelial cells of the vascular network of human adipose tissue, stem cells of human adipose tissue and collagen, the extraction of said extracellular matrix comprising a mechanical dissociation step; mixing said stromal vascular fraction and said extracellular matrix; culturing the mixture obtained in the preceding step, in suspension, in a cell proliferation medium; and induction of a differentiation of the stem cells of adipose tissue to obtain brown or beige adipocytes.

Advantageously, —the differentiation of the stem cells of adipose tissue is induced in a differentiation medium; —the mechanical dissociation step is a dissociation step which does not involve collagenase; —the mechanical dissociation step is a non-enzymatic dissociation step; —the extraction of the stromal vascular fraction and the extracellular matrix comprises the following steps: centrifugation of human adipose tissue to obtain at least two distinct fractions, a fraction A comprising a centrifuged extracellular matrix, and the mechanical stromal vascular fraction; and mechanical dissociation of the fraction A to obtain the extracellular matrix; —the method further includes a step of eliminating the blood cells present in the stromal vascular fraction; —the collagen of the extracellular matrix is structured collagen, which has a fibrillar organisation; —culturing the mixture of said stromal vascular fraction and said extracellular matrix comprises the following steps: transferring said mixture, in a sterile manner, into a suspension culture bag comprising the proliferation medium; amplifying said mixture to obtain an amplified mixture comprising cell clusters; —the amplified mixture comprising the cell clusters is the subject of a mechanical dissociation to obtain cell aggregates; and—the proliferation medium comprises a serum, a fibroblast growth factor and an insulin-like growth factor; and—the differentiation medium comprises Rosiglitazone and/or SB431542 and—the method further includes a cell sorting step aimed at sorting the stem cells expressing the surface marker DPP4.

According to a third object, the invention relates to an isolated extracellular matrix likely to be obtained according to the method defined above, comprising endothelial cells of the vascular network of human adipose tissue, stem cells of brown or beige adipocytes of human adipose tissue, and collagen.

According to a fourth object, the invention relates to a composition comprising a mixture of the extracellular matrix as above and a stromal vascular fraction comprising endothelial cells of the vascular network of adipose tissue and stem cells of human adipose tissue.

According to a fifth object, the invention relates to a kit comprising the composition as defined above, a cell proliferation medium, and a differentiation medium.

According to a sixth object, the invention relates to the use of the extracellular matrix as defined above or the composition as defined above for the screening and/or characterisation of pharmaceutical active ingredients.

According to a seventh object, the invention relates to brown or beige adipocytes resulting from a composition as above, for the use thereof in cell therapy, or for the treatment of metabolic disorders.

According to an eighth object, the invention relates to brown or beige adipocytes resulting from a composition as above, for the treatment of obesity.

According to a ninth object, the invention relates to a differentiation medium for the differentiation of stem cells of brown or beige adipocytes into brown or beige adipocytes, comprising an endothelial cell growth medium supplemented with serum, growth factors, rosiglitazone and SB431542 and, preferably, with foetal calf serum, fibroblast growth factors, insulin-like growth factors, vascular endothelial growth factors, ascorbic acid, rosiglitazone, T3, insulin, and SB431542.

### BRIEF DESCRIPTION OF FIGURES

The invention will be better understood on reading the following non-limiting description, drafted relative to the appended drawings, in which:

FIG. 1A schematically represents the steps which are required and sufficient for the extraction of an extracellular matrix and a stromal vascular fraction (steps 1 to 3), and the co-culture thereof (step 4), according to the invention;

FIG. 1B is a more detailed schematic representation of the method which allows the sequential extraction of extracellular matrices (M1-M4) and cell populations (C1-C3) of stromal vascular fraction (steps 1 to 5), and the co-culture thereof (step 6), according to the invention;

FIG. 1C is an illustration of products according to the invention, namely the product called ExAdEx-tissue, which results from the amplification of the mixture of the stromal vascular fraction and the extracellular matrix, and the product called ExAdEx-lobules, which results from the formation of aggregates, in accordance with an additional step of the method according to the invention;

FIG. 1D illustrates the steps for obtaining the product ExAdEx-lobules from the product ExAdEx-tissue according to the method of the invention;

FIGS. 2A, 2B, 2C, 2D, 2E, 2F, 2G, 2H, 2I, 2J and 2K are graphs which compare, respectively, the amounts expressed, in quantitative PCR, of the genes listed below in the products ExAdEx-lobules and ExAdEx-tissue: FABP4, PLIN1, Adiponectin, CD31, DPP4, ICAM1, PDGFRa, Inhibin beta A, MSCA1, IL1b (M1 macrophages), MRCI (M2 macrophages);

FIGS. 3A, 3B, 3C, 3D, 3E, 3F, 3G and 3H are photographs, obtained by confocal imaging, of products ExAdEx-lobules, highlighting, by immuno-fluorescent marking, in said products, respectively collagen I, collagen IV, fibronectin, laminin, elastin, DPP4, ICAM1 and CD31;

FIGS. 4A, 4B and 4C are figures representative of the presence, respectively, of the hypoxia marker CA9, in human adipocyte stem cells for five different proliferation media tested according to the method of the invention and PLIN1 and UCP1 in the differentiated stem cells, for five different proliferation media enriched with differentiation factors tested according to the method of the invention;

FIGS. 5A, 5B and 5C are optical microscopy images which show, respectively, that the differentiation medium allows a differentiation into adipocytes (Figure SA), that it is toxic for the endothelial cells in the presence of dexamethasone and IBMX (FIG. 5B), but that it is not, when these compounds are removed from said medium (FIG. 5C);

FIGS. 6A and 6B are figures which illustrate the expression of PLIN1 and UCP1 in different differentiation media according to the invention;

FIGS. 7A, 7B and 7C show images of fluorescence microscopy which allow visualising endothelial cells and different populations of stem cells of adipose tissue according to the presence of the marker CD31 (FIG. 7A), DPP4 (FIG. 7B) and ICAM1 (FIG. 7C) in the adipose tissue before the implementation of the method according to the invention (photographs on the left), after 20 days in the proliferation medium (photographs of the centre) and after 20 days in the proliferation medium then 20 days in the differentiation medium, except for ICAM1 (photographs on the right except for ICAM1);

FIGS. 8A and 8B show fluorescence microscopy images with labelling, by antibodies coupled to fluorochromes, DPP4 and ICAM1 proteins in the stromal vascular fractions and the extracellular matrix forming the fraction called “Endostem” fraction in these figures, before mixing according to the method according to the invention;

FIG. 9A shows, by fluorescence microscopy, that the matrix M2 is of the collagen-rich type; PicroSirius Red marked collagen (light grey) and marking of nuclei (white);

FIG. 9B shows, by fluorescence microscopy, that the matrix M3 is of the fibrous type; PicroSirius Red marked collagen (light grey and white fibres) and marking of nuclei (white);

FIG. 9C characterises, in microscopy, the fibrous type of the matrix M1;

FIG. 9D shows, by microscopy, that the matrix M2 is heterogeneous in terms of matrix types: fibrous type and collagen-rich type;

FIG. 9E is a microscopy image illustrating the fibrous type of the matrix M3;

FIG. 10A is a photograph of adipose tissue centrifuged from the fraction A after mechanical dissociation containing the matrix M4;

FIG. 10B highlights by fluorescence microscopy, in the matrix M4, of mature adipocytes by Oil Red 0 (light grey) colouring and a collagen-rich matrix by marking the type I collagen (very light grey);

FIG. 10C shows, by CD31 immunomarking, the capillary structures formed by CD31+ endothelial cells (white) in the M4 matrix; marking of nuclei (dark grey);

FIG. 10D illustrates the presence of the stem cell array of the PDGFRa+ adipose tissue (light grey dots) in the matrix M4; marking of nuclei (dark grey);

FIG. 11 shows, by incorporation of Edu, 5-ethylnyl 2′-deoxyuridine, in the nucleus of the cells in proliferation, that the endogenous cells, in the extracellular matrix of the invention are maintained in proliferation in the EGM+™ medium in suspension; nuclei (dark grey), proliferating cells (white) autofluorescence of the matrix (light grey);

FIG. 12 shows that stem cells of the exogenous adipose tissue, co-cultured with the extracellular matrix of the invention, form structures composed of these stem cells of adipose tissue and endogenous cells present in the matrix; image taken after 3 days of co-culture, nuclei (dark grey), collagen (light grey), exogenous stem cells of adipose tissue (light grey/white);

FIG. 13A shows the absence of proliferation of cells during the cell culture of the stem cells of adipose tissue and endothelial cells in suspension without extracellular matrix, from the stromal vascular fraction; image taken after 10 days of co-culture; nuclei (dark grey), nuclei of proliferating cells (very light grey);

FIG. 13B highlights a cell proliferation capacity of the stromal vascular fraction, in suspension, with the extracellular matrix of the invention; image taken after 10 days of co-culture; nuclei (dark grey), collagen matrix (light grey), proliferating cell nuclei by Edu marking (white);

FIG. 14A shows the level of expression of the endothelial cell marker CD31 in differentiated cell populations obtained by culture, in suspension, with (right) and without (left) the extracellular matrix of the invention;

FIG. 14B shows the level of expression of the adipocyte stem cell marker PDGFRa in the differentiated cell populations obtained by culture, in suspension, with (right) and without (left) the extracellular matrix of the invention;

FIG. 14C shows the level of expression of the mature adipocyte marker PLIN1 in the differentiated cell populations obtained by culture, in suspension, with (right) and without (left) the extracellular matrix of the invention;

FIG. 14D shows the level of expression of the mature adipocyte marker Adiponectin in the differentiated cell populations obtained by culture, in suspension, with (right) and without (left) the extracellular matrix of the invention;

FIGS. 15A and 15B are images which highlight the activation of the proliferation capacities of the method according to the invention. In FIG. 15A, an undissociated adipose tissue does not shows proliferating cells. In FIG. 15B, the composition shows proliferating cells, the nuclei of the proliferating cells being represented in white in this figure;

FIGS. 16A, 16B, 16C, 16D, 16E and 16F are images in which the proliferating cells, which are contained in the matrix called Endostem matrix, are marked, in the absence of the stromal vascular fraction (FIGS. 16A, 16C, 16E) and with the addition of this stromal vascular fraction (FIGS. 16B, 16D, 16F) after 20 days of culture in the proliferation medium;

FIGS. 17A, 17B, 17C, 17D, 17E and 17F illustrate the expression of dipeptidyl peptidase-4 (DPP4), which is concentrated in the isolated stromal vascular fraction, and the expression of ICAM1 and CD31, which is concentrated in the isolated matrix (FIGS. 17A, 17C, 17E), and the expression of DPP4, ICAM1 and CD31 in the amplified composition (FIGS. 17B, 17D, 17F);

FIG. 18 is composed of four photographs which illustrate the cell proliferation capacity (positive EdU) which has the marker DPP4 within the extracellular matrix of adipose tissue in the method according to the invention;

FIG. 19 is composed of photographs which illustrate the ability to differentiate into brown or beiges adipocytes of the cell populations show the marker DPP4 (three top photographs of the Figure) or which do not show this marker DPP4 (three bottom photographs);

FIGS. 20A and 20B are graphs which illustrate the ability to differentiate into brown or beige adipocytes of the cell populations, expressing DPP4 or not, obtained by quantitative PCR, in real time of the marker PLIN1 (FIG. 20A) and the marker UCP1 (FIG. 20B);

FIGS. 21A and 21B illustrate the presence of M1-type and M2-type macrophages respectively, in the amplified composition according to the invention;

FIG. 22 comprises a set of photographs which demonstrate the presence of certain proteins in the extracellular matrix according to the invention, and the preservation of a capillary network;

FIG. 23 shows the relative expression of the human UCP1, on day DO, i.e. before transplantation of the amplified product ExAdEx-tissue and on day D21, in the product ExAdEx-tissue, after transplantation in mice;

FIG. 24 shows three fluorescence microscopy photographs, the photograph on the left showing the absence of brown/beige adipocytes in the white adipose tissue before the implementation of the method of the invention, the photograph of the centre showing the presence of brown/beige adipocytes, specifically marked in light grey by the ex vivo expression, in an overweight individual, of the UCP1 protein, after the amplification and differentiation method, and the photograph on the right showing the presence of ex vivo brown/beige adipocytes, after implementation of the invention, in a patient, with severe obesity;

FIGS. 25A, 25B and 25C show the viability of stem cells of adipose tissue after purification of the stromal vascular fraction, directly after incubation in a 1×lysis buffer, named ACL, comprising ammonium chloride (FIG. 25A), after 24 hours of culture (FIG. 25B) and after 5 days under adherent culture conditions (FIG. 25C);

FIGS. 26A, 26B, 26C and 26D show the graphical results of the flow cytometry analysis of the number of viable and dead cells after different times of treatments in a 1×lysis buffer, named ACL, comprising ammonium chloride;

FIGS. 27A, 27B and 27C show the viability of endothelial cells of adipose tissue after purification of the stromal vascular fraction, directly after incubation in a 1×lysis buffer, named ACL, comprising ammonium chloride (FIG. 27A), after 24 hours of culture (FIG. 27B) and after 5 days under adherent culture conditions (FIG. 27C);

FIG. 28 shows the stem cell profile of adipose tissue, amplified according to the ExAdEx method of the invention, obtained by determination by flow cytometry of the cell surface markers;

FIGS. 29A and 29B show the profiles of the ASCs of ex vivo adipose tissue (FIG. 29A) and the amplified ExAdEx product (FIG. 29B) obtained by determining the cell surface markers obtained by flow cytometry;

FIGS. 30A, 30B and 30C show endothelial cells isolated from human adipose tissue which are cultured in the proliferation medium described in the invention (FIG. 30A), in the differentiation medium described in the invention (FIG. 30B) and in a non-optimised standard differentiation medium which comprises DMEM and serum (FIG. 30C).

FIG. 31 has a visualisation in white, of a functional vascular network of the composition described in the invention after transplantation in the Nude mice (4 weeks post-transplant); and

FIG. 32 shows a comparison of the viability and amplification capacities of the stem cells of brown or beige adipocytes of adipose tissue according to a method not comprising the addition of the stromal vascular fraction (A) and comprising the purification then the addition of the cells isolated from the infranatant, also named, in the invention, stromal vascular fraction (B).

### DETAILED DESCRIPTION OF THE INVENTION

Adipose tissue is provided to carry out the invention. It is in practice adipose tissue, for example white from individuals, for example, but not exclusively, overweight individuals, in particular obese and/or having metabolic disorders such as the type 2 diabetes and cardiovascular diseases.

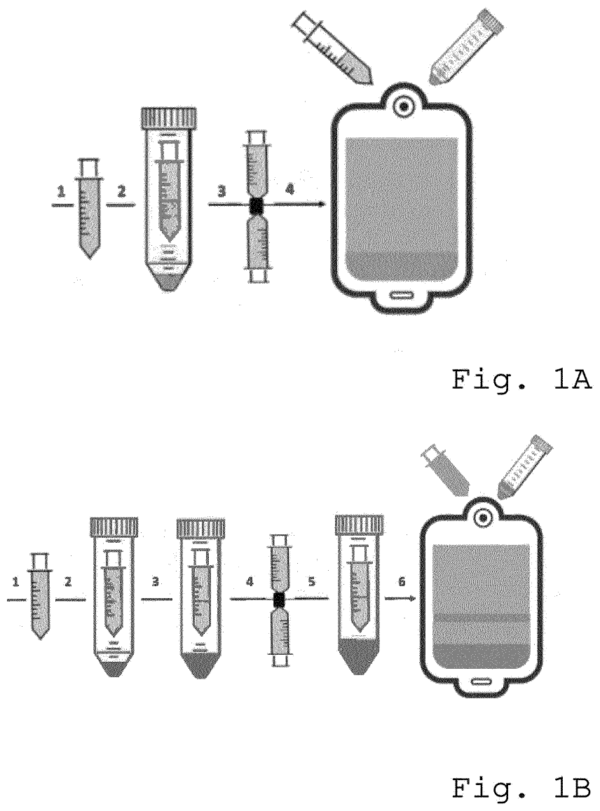

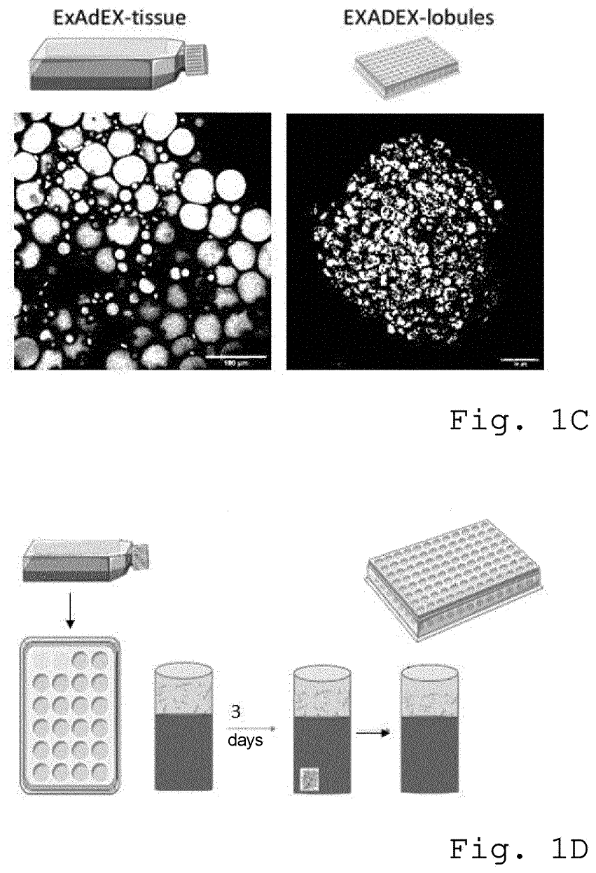

The first object of the invention is a method for the in vitro or ex vivo amplification of stem cells of brown or beige adipocytes from adipose tissue, for example white, human adipose tissue comprising the following steps: extracting, on the one hand, a stromal vascular fraction (FIG. 1A, steps 1-2 and FIG. 1B, steps 1-3 and step 5) called mechanical stromal vascular fraction from human adipose tissue comprising endothelial cells of the vascular network of human adipose tissue and stem cells of human adipose tissue and, on the other hand, an extracellular matrix of said human adipose tissue (FIG. 1A, step 3 and FIG. 1B, step 4), said extracellular matrix comprising endothelial cells of the vascular network of human adipose tissue, stem cells of human adipose tissue and collagen, the extraction of said extracellular matrix comprising a mechanical dissociation step (FIG. 1A, step 3 and FIG. 1B, step 4); mixing said stromal vascular fraction and said extracellular matrix (FIG. 1A, step 4 and FIG. 1B, step 6); and culturing the mixture obtained in the preceding step, in suspension, in a culture medium, namely a cell proliferation medium. This method is also referred to below as the “ExAdEx method” (for Ex vivo Adipocytes Expansion).

Within the meaning of the present invention, the term “stromal vascular fraction” means the cells present in a sample of human adipose tissue. This stromal vascular fraction includes endothelial cells of the vascular network of human adipose tissue and stem cells of human adipose tissue. The stromal vascular fraction according to the invention, also called infranatant, is advantageously derived from steps 2 and 3 described in FIG. 1B, and is rich in DPP4* cells.

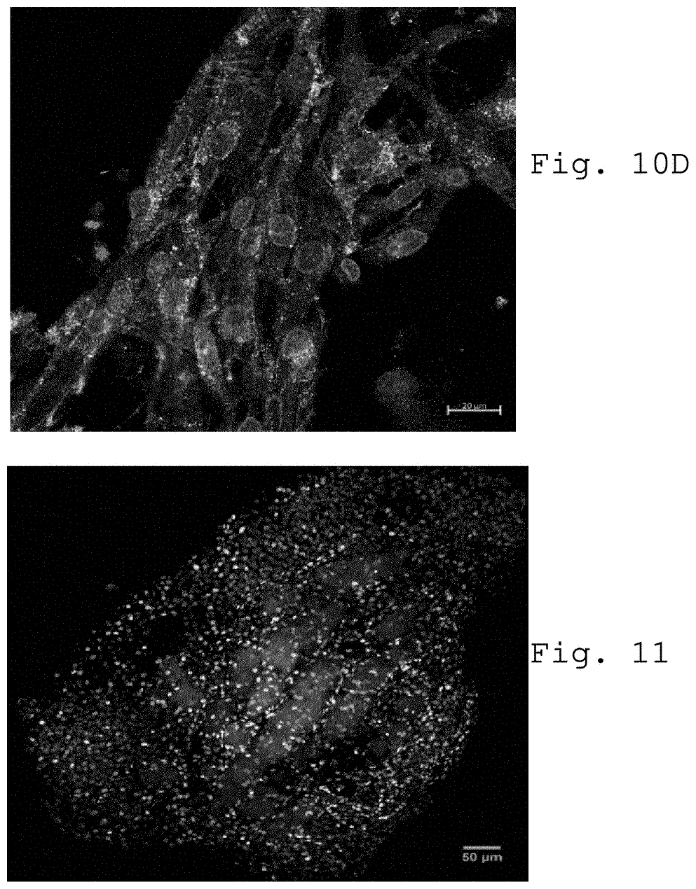

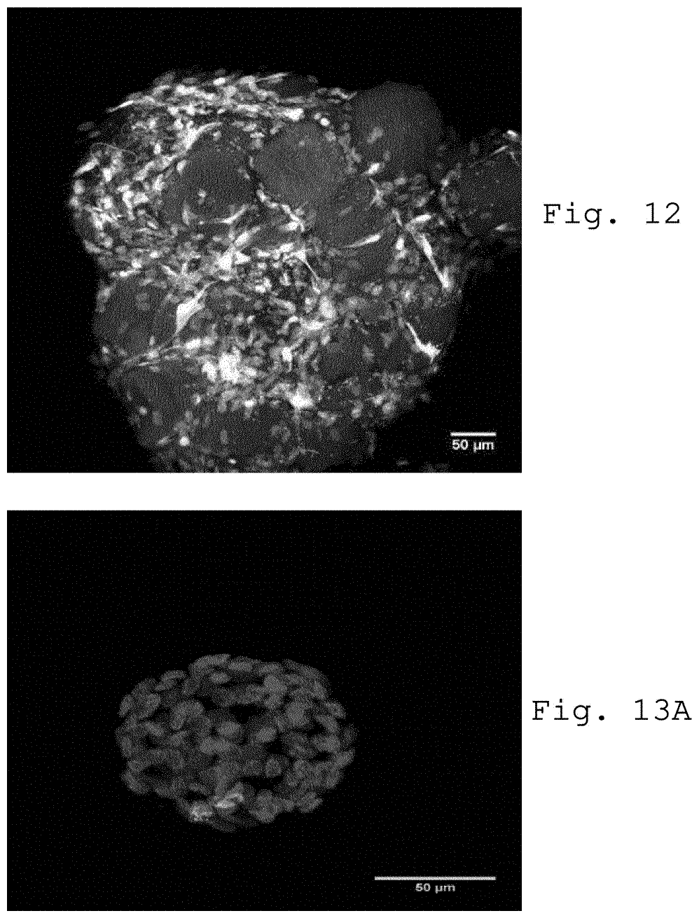

Within the meaning of the invention, the term “extracellular matrix” means a bioactive matrix, that is to say a matrix which comprises different proteins of adipose tissue (FIG. 22) and endogenous cells, comprising in particular endothelial cells (FIG. 10C), stem cells of adipose tissue (FIG. 10D), adipocytes and macrophages. FIG. 11 shows the presence of proliferating cells in the extracellular matrix (Edu marking in white). This extracellular matrix allows the 3D cell amplification, that is to say the proliferation of the cells in three dimensions. The extracellular matrix of the invention is also denoted hereinafter “EndoStem-Matrix”.

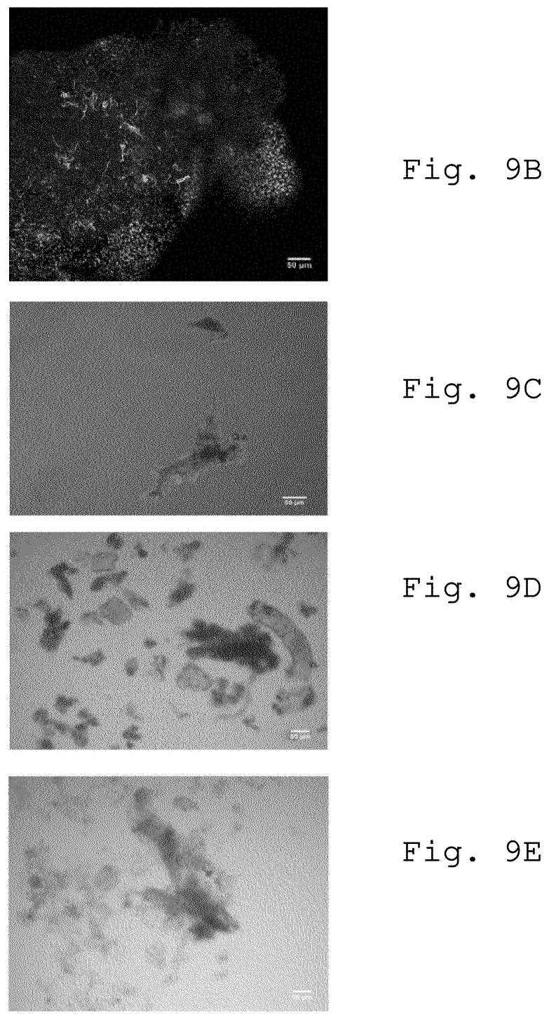

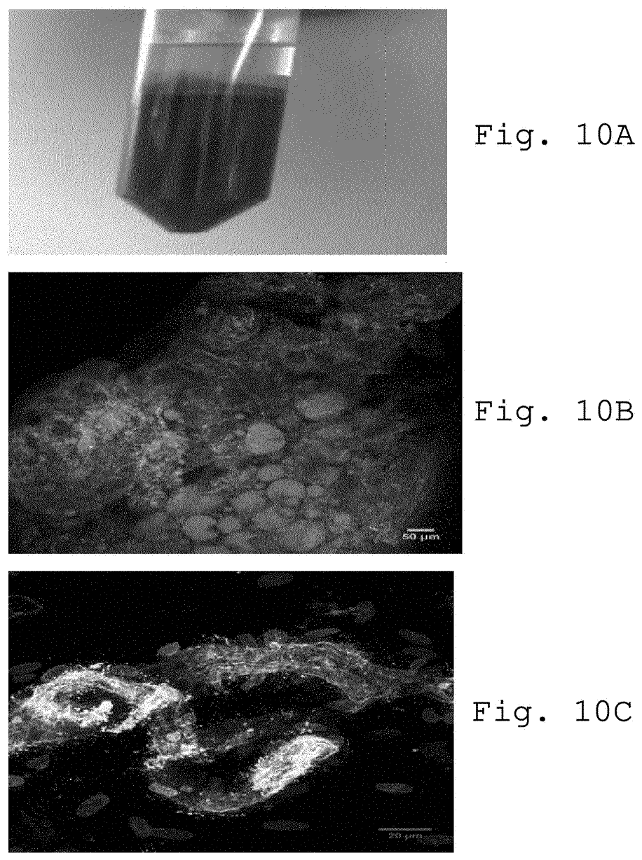

The proteins of the extracellular matrix of adipose tissue comprise collagen. This collagen is structured. It has a fibrillar organisation. It is in particular type I and type III collagen (see FIGS. 9A and 9B showing PicroSirius Red marked fibrillar collagen fibres and FIG. 10B which shows the type I collagen marked by anti-collagen antibody and observed by confocal microscopy). The proteins of the extracellular matrix of adipose tissue further comprise in particular fibronectin, elastin, laminin and type IV collagen (FIG. 22).

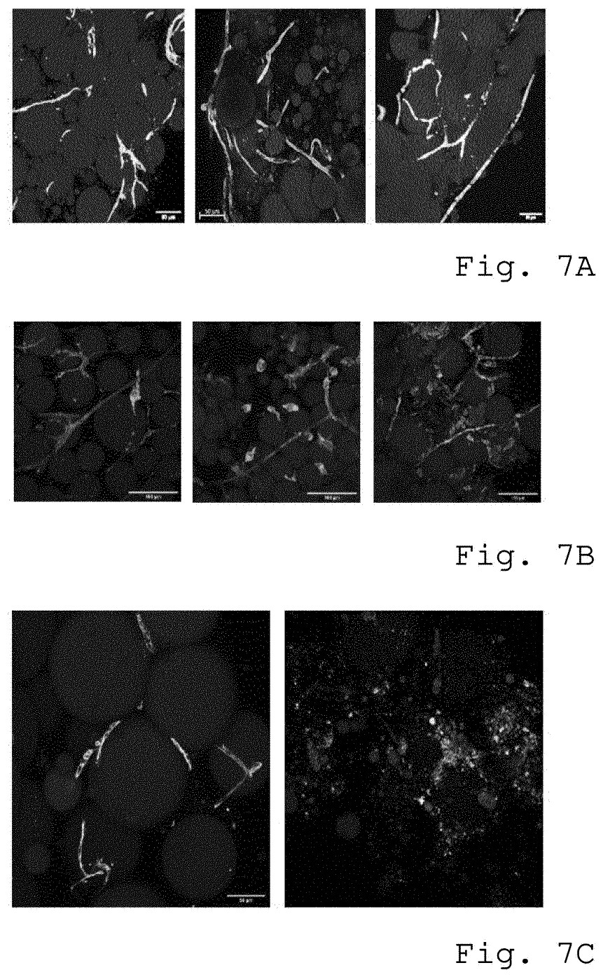

The extraction of the extracellular matrix comprises a non-enzymatic dissociation step, in particular the extraction of the extracellular matrix comprises a mechanical dissociation step. The “mechanical dissociation” of the invention allows keeping intact the structure of the extracellular matrix while an enzymatic digestion generally involves collagenase which digests it. The mechanical dissociation thus allows the maintaining the “vasculature”, as well as this is shown in FIG. 7A, in which the vascular network appears before dissociation in human adipose tissue in the photograph on the left and the vascular network after dissociation and amplification in the middle photograph. This mechanical dissociation further allows the maintenance of the microstructure of the extracellular matrix, which consequently has an organisation similar to the organisation of in vivo adipose tissue.

The extraction of the stromal vascular fraction and the extracellular matrix comprises the following steps: centrifugation of human adipose tissue to obtain at least two distinct fractions, a fraction A comprising a centrifuged extracellular matrix, and the stromal vascular fraction; and mechanical dissociation of the fraction A to obtain the extracellular matrix (FIG. 1A).

The human adipose tissue centrifugation step allows, in addition to removing oil, blood and anaesthetic liquid contained in the provided human adipose tissue. This step also allows removing the physiological fluid resulting from preliminary washings of provided human adipose tissue.

In a particular embodiment, the extraction of the stromal vascular fraction and the extracellular matrix comprises the following steps: centrifugation of human adipose tissue to obtain at least two distinct fractions, a fraction A comprising a centrifuged extracellular matrix and a fraction B comprising endothelial cells of the vascular network of human adipose tissue and stem cells of human adipose tissue; mechanical dissociation of the fraction A to obtain a fraction A′ comprising a dissociated extracellular matrix; centrifugation of the fraction A′ to obtain at least the extracellular matrix and a fraction B′ comprising endothelial cells of the vascular network of human adipose tissue and stem cells of human adipose tissue; and mixture of the fractions B and B′ to obtain the mechanical stromal vascular fraction (FIG. 1B).

In this embodiment, the step of centrifuging human adipose tissue further allows removing oil, blood and anaesthetic liquid contained in the provided adipose tissue. This step also allows removing the physiological fluid resulting from preliminary washings of provided human adipose tissue. The centrifugation of the fraction A′ also allows removing any oil and physiological fluid residues. This step of centrifuging the fraction A′ is optional.

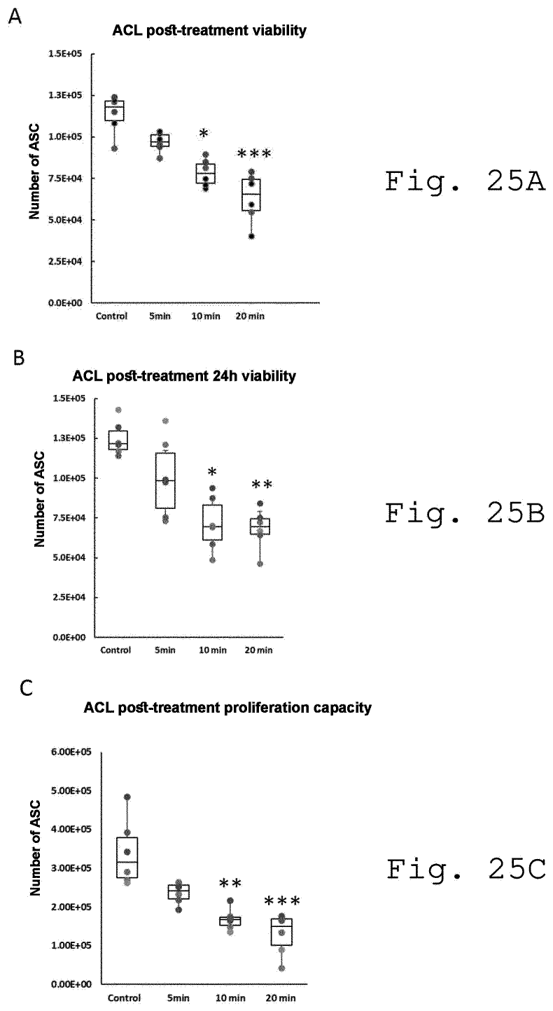

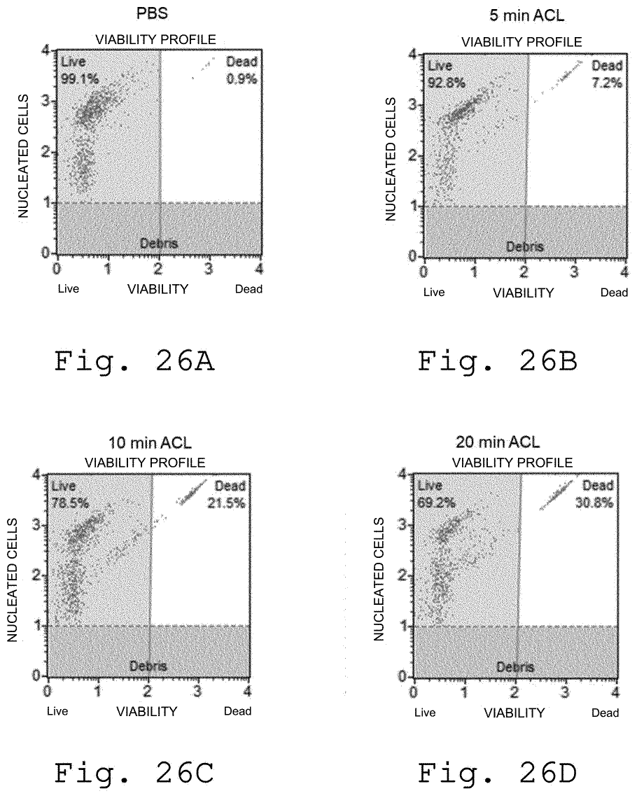

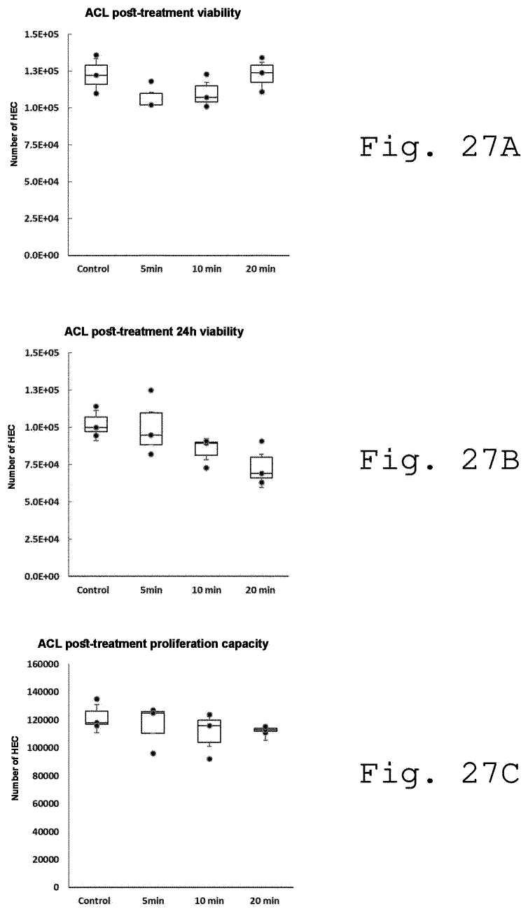

In a particular embodiment, the method according to the invention further includes a step of eliminating the blood cells. This is an elimination of erythrocyte-type blood cells, present in adipose tissue and in the different cell pellets, called mechanical SVF, obtained during the aforementioned steps of the mechanical dissociation method called ExAdEx. This elimination step is carried out during an incubation of adipose tissue and/or cell pellets in a 1×lysis buffer comprising ammonium chloride (“Ammonium chloride lysing solution”, Becton Dickinson™—named ACL) diluted in sterilised water, in a buffer: sample ratio, ranging from 1:1 to 1:10, at a temperature comprised between 4 and 37° C. and during an incubation time ranging from 5 minutes to 30 minutes. This step allows the lysis of erythrocytes. FIGS. 25A, 25B and 25C as well as FIGS. 26A, 26B, 26C and 26D show the effect, on the stem cells of adipose tissue, of the LCD treatment. This results in an incubation in the ACL, over a time period which is greater than 5 minutes, damages the viability and proliferation capacities of the stem cells contained in the infranatant. Moreover, FIGS. 27A, 27B and 27C illustrate the absence of the effect of the incubation in the ACL on the viability and proliferation capacities of the endothelial cells of adipose tissue which are contained in the infranatant.

The culture of the mixture of the stromal vascular fraction and said extracellular matrix comprises the following steps: transferring said mixture, in a sterile manner, into a suspension culture bag comprising the proliferation medium; and amplifying said mixture forming cell clusters.

The transfer in a “sterile manner”, within the meaning of the invention, is a transfer, preferably, carried out in a closed system. This transfer, in a sterile manner, allows avoiding the presence of contaminants during the cell culture. The mechanical dissociation of the cell clusters formed during amplification does not require the opening of the system, thus avoiding the exposure of the cell products to a contamination of the culture by the elements of the environment.

In one embodiment, the proliferation medium, in the suspension culture bag, is an EGM+™ medium. This proliferation medium comprises the base medium for endothelial cell proliferation (EGM) enriched with Epidermal Growth Factor (EGF), Basic Growth Factor (FGF2), Insulin-like Growth Factor, Vascular Endothelial Growth Factor 165, ascorbic acid, heparin and hydrocortisone (EGM+). The EGM+ medium also allows the amplification of adipocyte stem cells without altering their ability to differentiate into adipocytes.

The method of the invention allows an amplification of the number of stem cells of adipose tissue with an amplification factor which is greater than 10, advantageously greater than 20, in particular greater than 30, preferably greater than 35. The amplification factor is the ratio between the number of cells obtained after culture of the isolated SVF in the presence of said extracellular matrix and the number of cells before the invention. In a particular embodiment described in Example 2, the method of the invention has an amplification factor from 36 in 8 days.

According to a second object, the invention relates to a method for the in vitro or ex vivo amplification of the stem cells of brown or beige adipocytes, comprising the following steps: in vitro or ex vivo amplification of stem cells of human adipose tissue as defined above; and induction of a differentiation of the stem cells of adipose tissue to obtain brown or beige adipocytes. In other words, according to a second object, the invention relates to a method for in vitro or ex vivo obtaining brown or beige adipocytes comprising the following steps: in vitro or ex vivo amplification of stem cells of human adipose tissue as defined above; and induction of a differentiation of the stem cells of adipose tissue to obtain brown or beige adipocytes.

More specifically, the method for the in vitro or ex vivo amplification of brown or beige adipocytes therefore comprises the following steps: extracting, on the one hand, a stromal vascular fraction from a human adipose tissue comprising endothelial cells of the vascular network of human adipose tissue and stem cells of human adipose tissue and, on the other hand, an extracellular matrix of said human adipose tissue, said extracellular matrix comprising endothelial cells of the vascular network of human adipose tissue, stem cells of human adipose tissue and collagen; mixing said mechanical stromal vascular fraction and said extracellular matrix; culturing the mixture obtained in the preceding step, in suspension, in a cell proliferation medium; and induction of a differentiation of the stem cells of adipose tissue to obtain brown or beige adipocytes. In other words, the method for in vitro or ex vivo obtaining brown or beige adipocytes therefore comprises the following steps: extracting, on the one hand, a stromal vascular fraction from a human adipose tissue comprising endothelial cells of the vascular network of human adipose tissue and stem cells of human adipose tissue and, on the other hand, an extracellular matrix of said human adipose tissue, said extracellular matrix comprising endothelial cells of the vascular network of human adipose tissue, stem cells of brown or beige adipocytes of human adipose tissue and collagen; mixing said stromal vascular fraction and said extracellular matrix; culturing the mixture obtained in the preceding step, in suspension, in a cell proliferation medium; and induction of a differentiation of the stem cells of brown or beige adipocytes of adipose tissue to obtain brown or beige adipocytes.

The method for the in vitro or ex vivo amplification of differentiated cells comprising the steps related to the in vitro or ex vivo amplification of stem cells of adipose tissue, the details given above for the method for the in vitro or ex vivo amplification of stem cells of adipose tissue also apply for the method for the in vitro or ex vivo amplification of differentiated cells. In other words, the method for in vitro or ex vivo obtaining brown or beige adipocytes comprising the steps related to the in vitro or ex vivo amplification of stem cells of brown or beige adipocytes of adipose tissue, the details given above for the method for the in vitro or ex vivo amplification of stem cells of brown or beige adipocytes of adipose tissue also apply for the method for in vitro or ex vivo obtaining brown or beige adipocytes.

In particular, the extraction of the extracellular matrix comprises a non-enzymatic dissociation step, in particular the extraction of the extracellular matrix comprises a mechanical dissociation step.

In one embodiment, the extraction of the stromal vascular fraction and the extracellular matrix comprises the following steps: centrifugation of human adipose tissue to obtain at least two distinct fractions, a fraction A comprising a centrifuged extracellular matrix, and the stromal vascular fraction; and mechanical dissociation of the fraction A to obtain the extracellular matrix.

In another embodiment, the extraction of the stromal vascular fraction and the extracellular matrix comprises the following steps: centrifugation of human adipose tissue to obtain at least two distinct fractions, a fraction A comprising a centrifuged extracellular matrix, and a fraction B comprising endothelial cells of the vascular network of human adipose tissue and stem cells of human adipose tissue; mechanical dissociation of the fraction A to obtain a fraction A′ comprising a dissociated extracellular matrix; centrifugation of the fraction A′ to obtain at least the extracellular matrix and a fraction B′ comprising endothelial cells of the vascular network of human adipose tissue and stem cells of human adipose tissue; and mixture of the fractions B and B′ to obtain the stromal vascular fraction.

The collagen of the extracellular matrix comprises type I collagen and type III collagen revealed by Picrosirius Red staining (see FIG. 9A and FIG. 9B).

In a particular embodiment of the method according to the invention, said method further includes a cell sorting step aimed at sorting the stem cells expressing the surface marker DPP4, also called CD26. A possible variant consists in selecting/sorting the cells expressing CD26/DPP4, mainly before the method called ExAdEx method, after mixing the stromal vascular fraction and the extracellular matrix, but before culturing said mixture, in suspension, in the cell proliferation medium. The objectives are in particular to enrich the product used in precursor cells of brown or beige adipocytes and, on the other hand, to homogenise and standardise the composition of the cell product to be amplified.

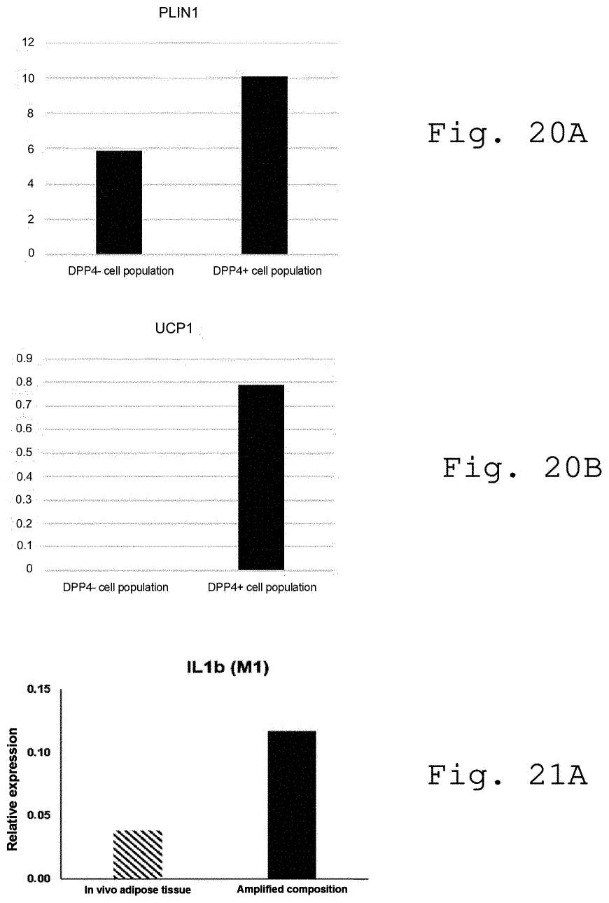

Indeed, a sorting of the stem cells of human adipose tissue sorted according to the presence of the surface marker CD26 has highlighted that the cells expressing the surface marker CD26 have the ability to be differentiated preferably in brown/beige adipocytes. FIG. 20A shows that the DPP4+ and DPP4—populations all have the ability to be differentiated into adipocytes according to the expression of the marker PLIN1 (FIG. 20A). However, only the DPP4+ population is differentiated into adipocytes expressing the brown/beige adipocyte marker UCP1 (FIG. 20B). An illustration in FIG. 19 shows by fluorescence imaging of the UCP1 protein than the UCP1 protein is only expressed in the stem cells of adipose tissue expressing DPP4+ and differentiated in an adipogenic medium. The use of the product, which is obtained after culturing the sorted cells, mainly relates to the cell therapy applications of obesity and metabolic diseases, however in vitro applications of this type of controlled composition product are also possible. The methodological modalities of cell sorting are based on the DPP4 specific antibody binding to identify the cells expressing this protein. A first separation technique consists in using antibodies coupled with fluorochromes, sorting takes place on an automated cell sorting platform (Aria III™ type, BD) which is a high throughput cytometer/cell sorter capable of separating at least four different cell populations. The other method relates to the immuno-magnetic cell sorting, based on the use of antibodies coupled with magnetic balls which allow the retention of the cells of interest in a magnetic field (CliniMACs™ type technology, Miltenyi Biotec™). These two techniques make it possible to allow achieve high degrees of homogeneity of the cell product of interest in the final product (>95%). These techniques are all compatible with sorting carried out under GMP conditions for cell therapy applications subject to having adapted platforms (Automate CliniMacs™ for the magnetic sorting, GMP sorting Cytometer).

In one embodiment of the invention, the culture of the mixture of said stromal vascular fraction and said extracellular matrix comprises the following steps: transferring said mixture, in a sterile manner, into a suspension culture bag comprising the culture medium; amplifying said mixture forming cell clusters;

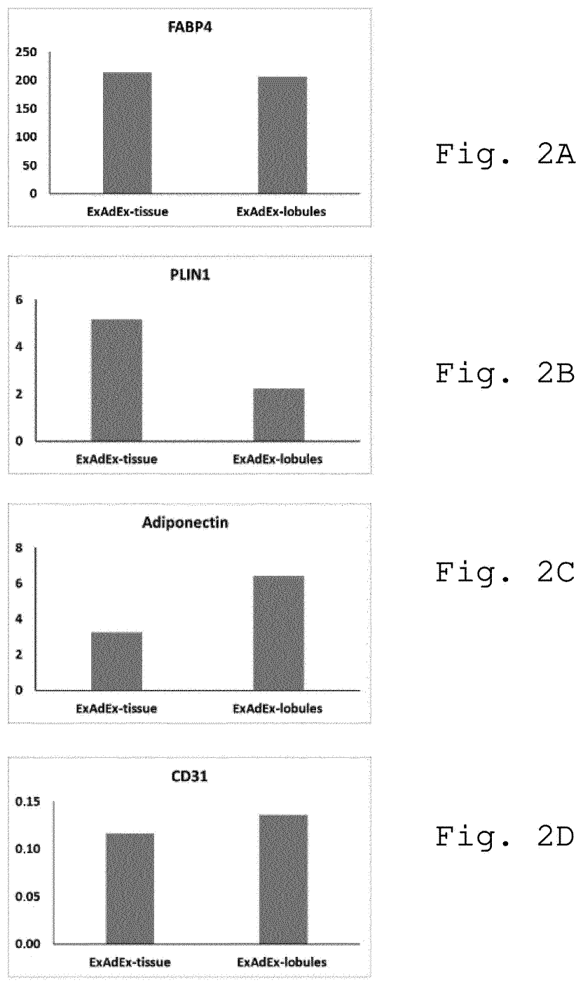

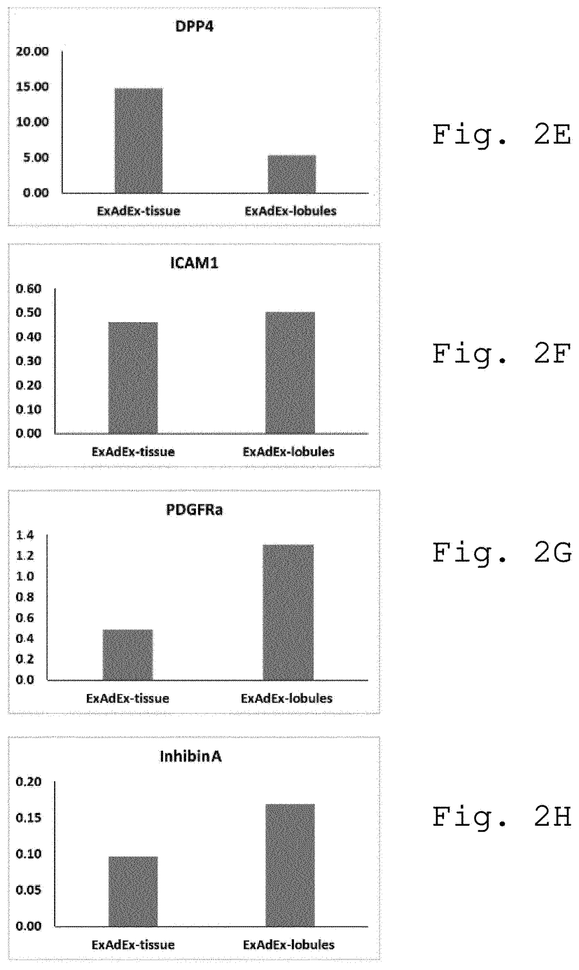

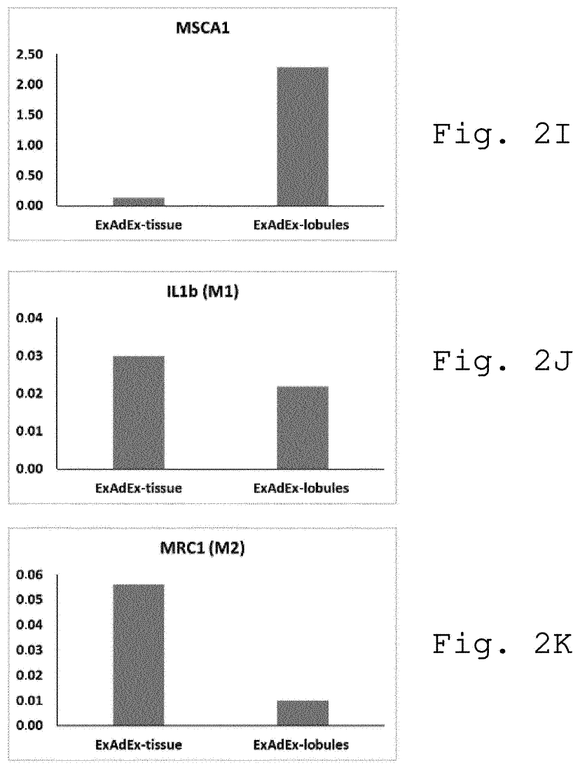

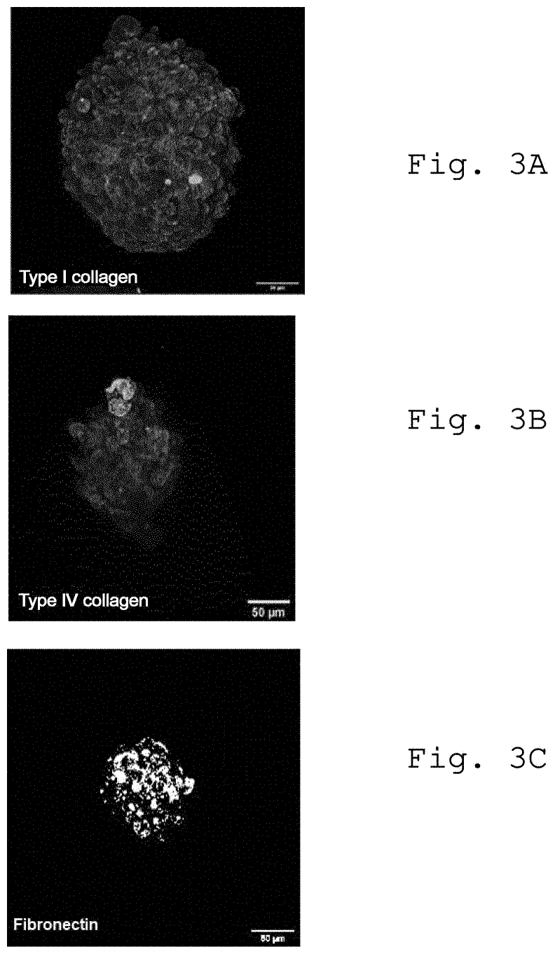

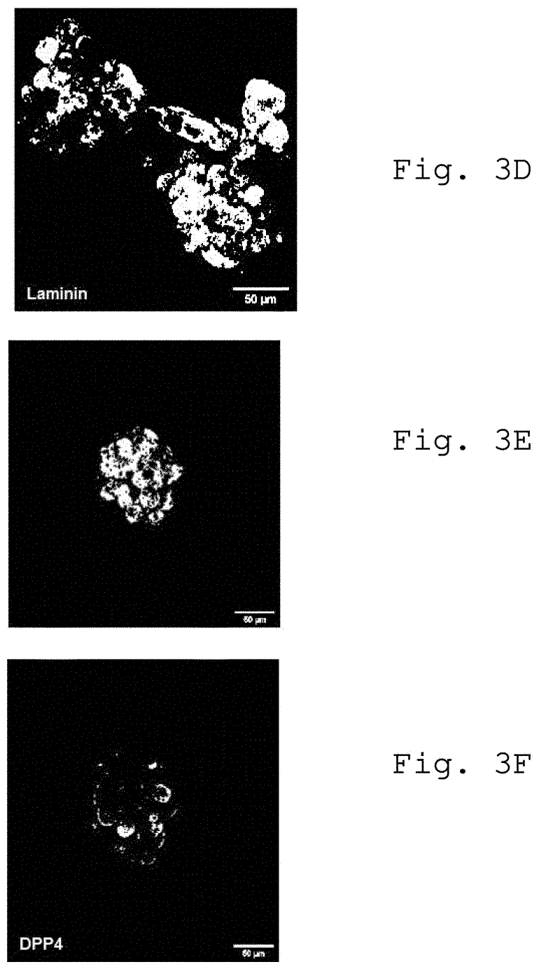

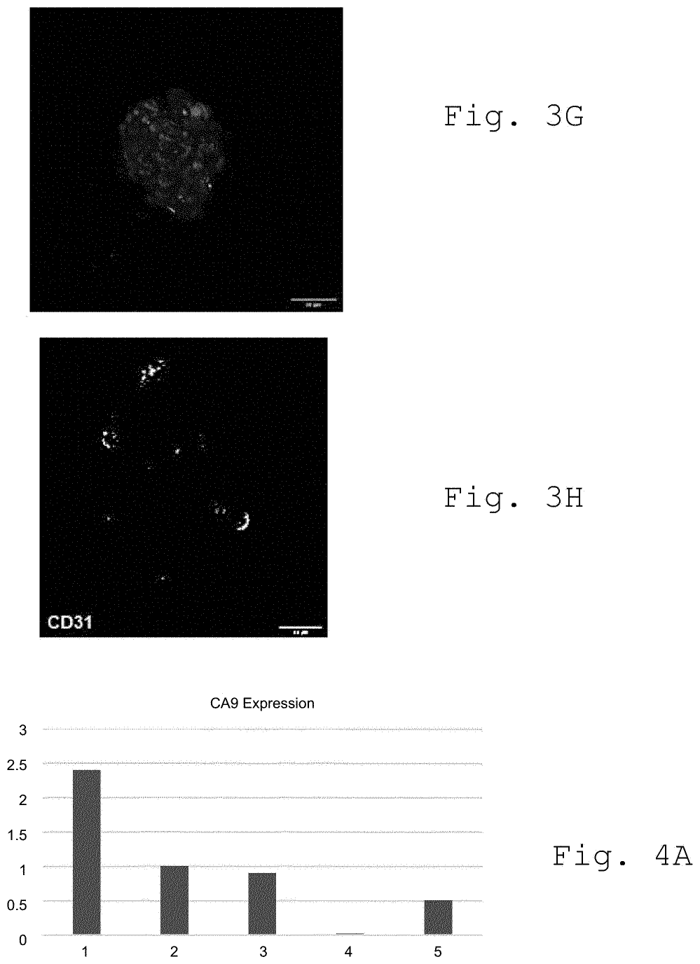

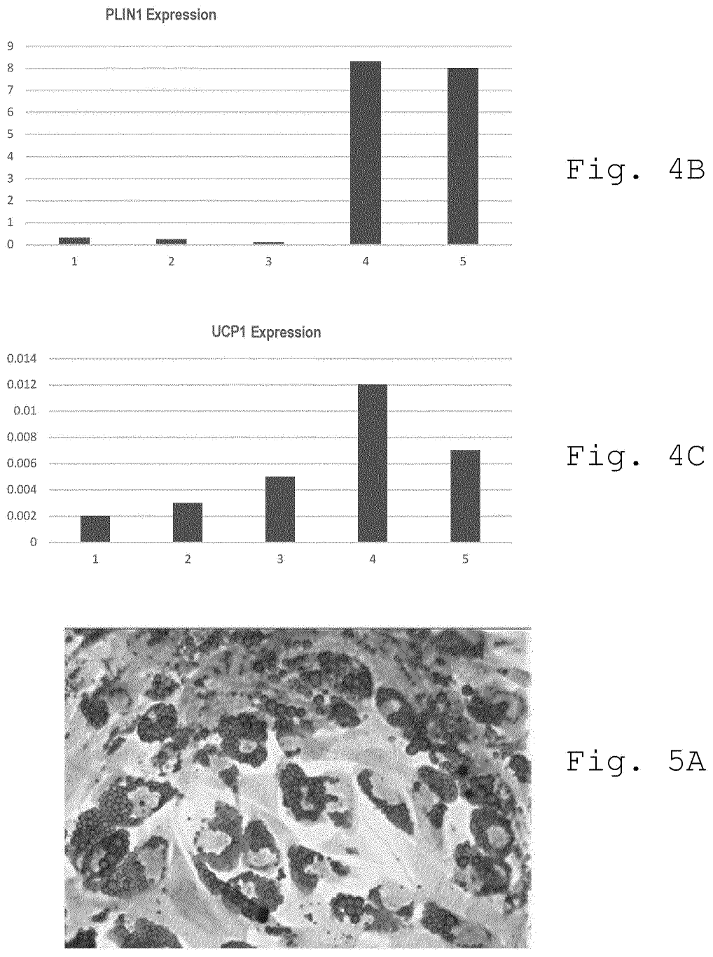

The product resulting from the amplification of the mixture of the mechanical SVF fraction and the matrix fraction can be referred to as “ExAdEx-tissue”. The product resulting from the formation of aggregates in an additional step at the end of the method, can be referred to as “ExAdEx-lobules”. These products are schematised in FIG. 1C. The product EXADEX-lobules is a product which can be obtained in an additional step of the method according to the invention. At the end of the co-culture step, the product is amplified in the EGM+ proliferation medium in culture flasks, as previously described. It then constitutes the amplified product ExAdEx-tissue. As illustrated in FIG. 1D, this amplified product is transferred into 6 well, 12 well or 24 well non-adherent ULA (Ultra-Low Attachment) culture plates, in the proliferation medium, in particular EGM+ proliferation medium, with or without stirring. A formation of aggregates is then observed. These include all the characteristics described in the product ExAdEx-tissue, after 3 to 10 days, under the described conditions. These characteristics are defined by the presence of stem cells of human adipose tissue, endothelial cells and the components of the extracellular matrix. This product is also characterised by the presence of mature adipocyte cells and macrophages. Among the molecular markers, and as shown in FIGS. 2A to 2K, the markers CD31, DPP4, ICAMI, FDGFRA, PLNI1, ADIPONECTIN, FABP4, IL1B and MRCI1 are found. These figures show a comparison of the expression of the previously mentioned markers, found in the products ExAdEx-tissue and ExAdEx-lobules in a comparable manner. Only the expression of MSCA1 is different between the two products and constitutes the molecular signature of the ExAdEx-lobules compared to the product ExAdEx-tissue. A characterisation of the proteins present in the ExAdEx-lobules was also made by confocal fluorescence microscopy and shows the presence of the main proteins of the extracellular matrix of adipose tissue, in particular type I collagen (FIG. 3A) and type IV collagen (FIG. 3B), but also fibronectin (FIG. 3C), laminin (FIG. 3D), elastin (FIG. 3E) and markers of stem cells of adipose tissue DPP4 (FIG. 3f), ICAM1 (FIG. 3G), as well as endothelial cell markers CD31 (FIG. 3H). The produced units are of sizes which are more homogeneous than the product ExAdEx-tissue and are adapted to the standards used by the pharmaceutical industries in the context of screening of molecules. The product ExAdEx-lobules is also better adapted for syringe injection in the context of a cell therapy. These ExAdEx-lobules units can be produced from the product ExAdEx-tissue at different amplification times, for example from 10 days of amplification and up to 40 days. These aggregates, once formed, no longer possess amplification capacities, but have a viability in culture greater than 10 days. The ExAdEx-lobules units can be maintained in the product range in white adipocytes or induced in differentiation or differentiated in brown or beige adipocytes.

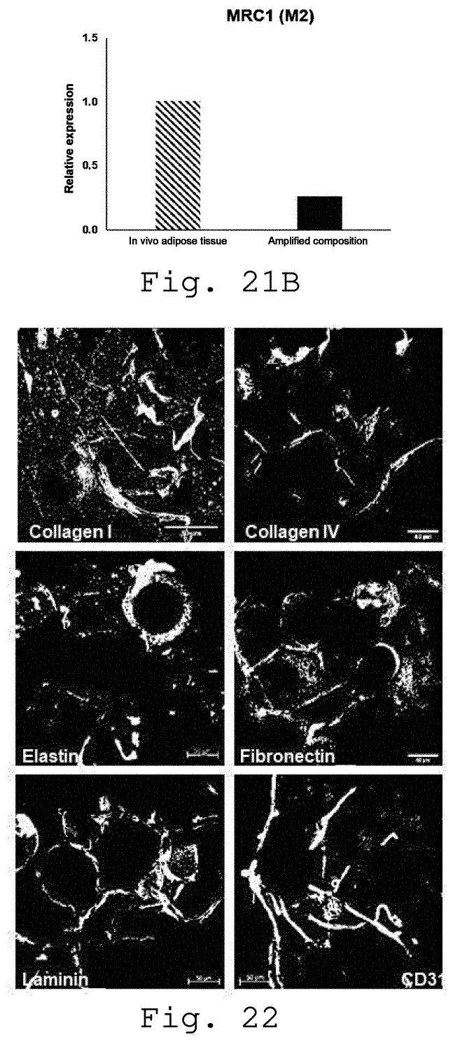

Ultimately, the method according to the invention aims at amplifying the brown or beige adipocyte stem cells (hASCs), maintained in a 3D active extracellular matrix, then to differentiate them into brown or beige adipocytes while allowing maintaining viable cells of the microenvironment of the adipose tissue namely the endothelial cells (hECs) and M1 and M2 macrophages (FIGS. 21A, 21B). To this end, culture media have been developed, because the culture medium conventionally used for the proliferation and differentiation of the hASCs is toxic for the hECs and the culture medium conventionally used for the proliferation of hECs is inhibitor of the hASC differentiation. Two culture media have therefore been established: a proliferation medium, which allows the proliferation of hASCs and a differentiation medium, which allows the differentiation of the amplified hASCs into adipocytes expressing the marker UCP1. The originality of these two culture media is that they are also compatible with the maintenance of the endothelial cells of human adipose tissue.

The medium referenced DMEM (Dulbecco™ Modified Eagle Medium), comprising 10% FCS (Foetal Calf Serum) is the reference medium for the proliferation of hASCs. However, this medium does not allow maintaining viable the endothelial cells. In order to determine the proliferation medium, five mediums, which are generally marketed for the proliferation of human endothelial cells, were tested. They are the following media:
No. 1: Endo-BM EPC: Marketed by the PrepoTech™ company (product reference: Cat: GS-EPC)
No. 2: Endo-BM MacroV Marketed by the PrepoTech™ company (product reference: Cat: GS-MacroV)
No. 3: Endo-BM MicroV Marketed by the PrepoTech™ company (product reference: Cat: GS-MicroV)
No. 4: EGM+ Marketed by the Promocell™ company (product reference: Cat: CC-22011 plus C-39216)
No. 5 MV2 Marketed by the Promocellcompany (product reference: Cat: CC-39226 plus C-22022B)

In 2D culture, these five media allow the proliferation of the stem cells of human adipose tissue with the same efficiency as the reference medium. However, the proliferation medium No. 4, namely EGM+, was selected because, in particular, unlike the other tested media, EGM+ does not induce cellular hypoxia, followed by the hypoxia marker CA9, when the hASCs are in 3D suspension, as shown in FIG. 4A.

The composition of the EGM+ proliferation medium is the following: Endothelium Cell Growth Medium (growth of endothelial cells) supplemented with: 2% FCS; 5 ng/ml Epidermal Growth Factor (EGF); 10 ng/ml Fibroblast Growth factor (FGF2—Fibroblast Growth Factor 2); 20 ng/ml long R3 Insulin like Growth Factor-1 (IGF-1); IF/ml Vascular Endothelial Growth Factor (VEGF) 165; 1 μg/ml Ascorbic acid; 22.5 μg/ml Heparin; 0.2 μg/ml Hydrocortisone.

In order to determine the differentiation medium, different media were tested. These media were enriched with adipogenic factors then tested for the differentiation of the hASCs. The results indicate that: unlike the media No. 1 to 3, the EGM+, supplemented with adipogenic factors, allows an adipocyte differentiation, determined by the expression of the adipocyte marker PLIN1, as shown in FIG. 4B. However, the expression of the specific marker UCP1 of the brown/beige adipocytes is very low, as well as this is shown in FIG. 4C. This first EGM+ differentiation medium 4 is therefore not optimised. It should also be noted that the medium n° 5 is an option to the EGM+ which has not been pursued further.

The composition of the non-optimised proliferation medium is therefore advantageously that described above, supplemented with adipocyte differentiation factors, namely: EGM supplemented with: 2% FCS; 5 ng/ml EGF; 10 ng/ml FGF2; 20 ng/ml long R3 1GF; 0.5 ng/ml VEGF factor 165; 1 μg/ml ascorbic acid; 22.5 μg/ml Heparin; 0.2 μg/ml Hydrocortisone: 2 μM Rosiglitazone; 1 nM T3; 2.5 μg/ml Insulin; 0.25 M Dexamethasone; 500 μM IBMX, a non-specific phosphodiesterase inhibitor.

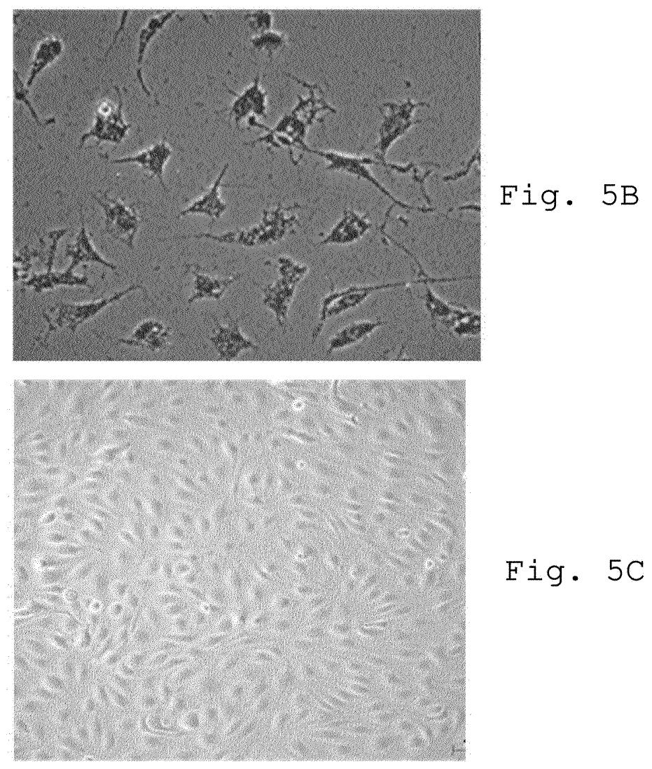

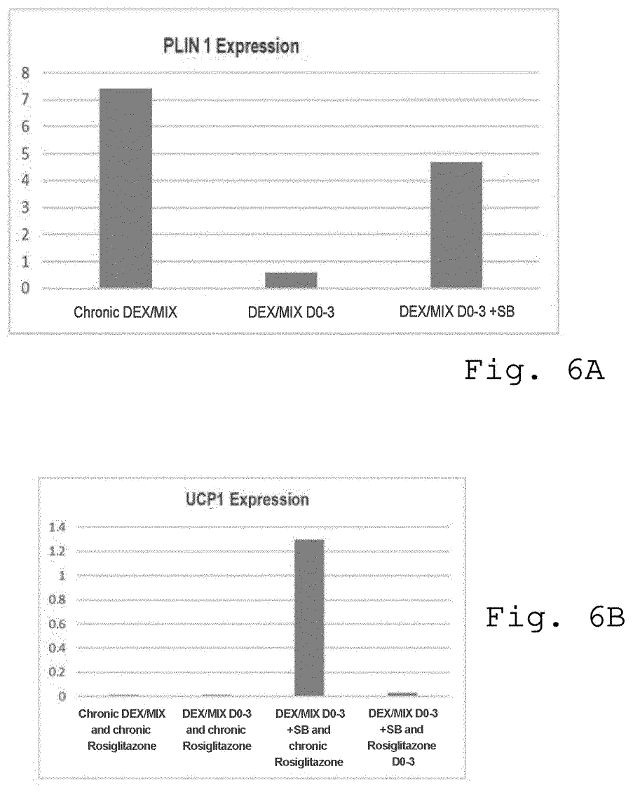

The aforementioned differentiation medium, which is not optimised, is advantageously optimised in the following manner. First of all, with the removal of EGF and hydrocortisone to increase the differentiation of hASCs: both compounds are described in the literature as being able to inhibit the expression of UCP1. Then, with the removal of Dexamethasone and IBMX to maintain the viability of the hECs: as shown in FIG. 5A, the differentiation medium allows a differentiation into adipocyte, but is toxic to hECs (Endothelial Cells—FIG. 5B). However, the removal of Dexamethasone and IBMX of the differentiation cocktail after the first three days allows maintaining the viability of the hECs (FIG. 5C). Then again, with the addition of the compound SB431542 to compensate for removal of Dexamethasone and IBMX for the differentiation into adipocytes: the impact of the removal of dexamethasone and IBMX from the differentiation cocktail of after the first three days is a decrease in the differentiation into adipocytes (PLIN1). The addition of the TGFb pathway inhibitor, SB431542, allows restoring the differentiation as shown in FIG. 6GA. SB431542 has no consequence upon the viability of hECs. Finally, the addition of SB431542 and the maintenance of Rosiglitazone allows the expression of UCP1, as shown in FIG. 6B. In addition, the presence of Rosiglitazone is particularly advantageous because the expression is very low if Rosiglitazone is only present for the first three days.

The final composition of the optimised differentiation medium is thus advantageously the following: EGM supplemented with: 0.1% to 5%, preferably 2% FCS; 2 ng/ml to 20 ng/ml, preferably 10 ng/ml FGF2; 10 ng/ml to 30 ng/ml, preferably 20 ng/ml long R3 IGF-1; 0.1 ng/ml to 1 ng/ml, preferably 0.5 ng/ml VEGF 165; 0.5 μg/ml to 2 μg/ml, preferably 1 μg/ml ascorbic acid; 0.5 μM to 4 μM preferably 2 μM Rosiglitazone; 0.5 nM to 10 μM, preferably 1 nM T3; 0.5 μg/ml to 10 μg/ml preferably 2.5 μg/ml Insulin; 0.1 μM to 0.500 μM, preferably 0.25 μM Dexamethasone; 100 μM to 800 μM, preferably 500 μM 3-isobutyl-1-methylxanthine (IBMX); 1 μM to 10 μM, preferably 5 μM SB431542.

This differentiation medium is a first differentiation medium, which includes dexamethasone and IBMX. However, these compounds are however only present for about the first three days of the differentiation. A second differentiation medium is used whose composition is consistent with that aforementioned of the first differentiation medium, but which does not include dexamethasone and IBMX.

The final composition of this second differentiation medium is therefore for example the following: 0.1% to 5%, preferably 2% FCS; 2 ng/ml to 20 ng/ml, preferably 10 ng/ml FGF2; 10 ng/ml to 30 ng/ml, preferably 20 ng/ml long R3 IGF-1; 0.1 ng/ml to 1 ng/ml, preferably 0.5 ng/ml VEGF 165; 0.5 μg/ml to 2 μg/ml, preferably 1 μg/ml ascorbic acid; 0.5 μM to 4 μM preferably 2 Mm Rosiglitazone; 0.5 nM to 10 nM, preferably 1 nM T3; 0.5 μg/ml to 10 μg/ml preferably 2.5 μg/ml Insulin; 1 μM to 10 μM, preferably 5 μM of SB431542.

Moreover, possibly, the first and/or second differentiation media comprise 5 μg/ml to 50 μg/ml, for example 22.5 μg/ml heparin and 1 μM to 20 μM, for Example 10 μM of Y27632.

As shown in FIG. 7A, hECs are always detectable after 20 days in the composition of the proliferation medium then 20 more days in the composition of the differentiation medium. In this Figure, the ECs are visualised by expression of the marker CD31. The left photo is a photo of the adipose tissue before the implementation of the method according to the invention. The centre photo is a photo of this tissue after 20 days in the proliferation medium. The right photo is a photo of this tissue after 20 days in the proliferation medium then 20 days in the differentiation medium. FIG. 7B also shows that the DPP4 cells are always detectable after 20 days in the composition of the proliferation medium then 20 more days in the composition of the differentiation medium. This is also valid for the adipocyte precursors ICAM1 which are always detectable in the amplified tissue after 20 days of co-culture (FIG. 7C).

The factors which, in the differentiation medium, cannot be required or used in other conditions: —Y27632 is reported as increasing the cell viability in suspension; —heparin; and —the adipogenic factors can be used at other concentrations.

According to an exemplary embodiment of the invention, the differentiation of the stem cells of brown or beiges adipocytes of adipose tissue into brown or beige adipocytes is induced in vivo. Example 4 below indeed demonstrates that the product amplified according to the method of the invention allows a differentiation of the stem cells of brown or beige adipocytes of adipose tissue into brown or beige adipocytes, after transplantation in Nude mice.

According to a third object, the invention relates to an isolated extracellular matrix likely to be obtained according to the method defined above, comprising endothelial cells of the vascular network of human adipose tissue, stem cells of brown or beige adipocytes of human adipose tissue and collagen. In other words, the extracellular matrix is extracted during the extraction step of the method of the invention, and therefore comprises all characteristics of the extracellular matrix described above.

Collagen is type I structured collagen, and type III collagen (FIG. 9A and FIG. 9B). The extracellular matrix further comprises in particular fibronectin (FIG. 22).

According to a fourth object, the invention relates to a composition comprising the mixture of the extracellular matrix and of the stromal vascular fraction as defined above, the extracellular matrix comprising endothelial cells of the vascular network of human adipose tissue, stem cells of adipose tissue, and collagen, and the stromal vascular fraction comprising endothelial cells of the vascular network of adipose tissue and stem cells of brown or beige adipose tissue.

In other words, the composition comprises the extracellular matrix which is extracted during the extraction step of the method of the invention, as well as the stromal vascular fraction which is extracted during this same extraction step of the method of the invention. The extracellular matrix of the composition therefore comprises all characteristics of the extracellular matrix described above. And the stromal vascular fraction of the composition therefore comprises all characteristics of the stromal vascular fraction described above.

Collagen is type I collagen and type III collagen. The extracellular matrix further comprises fibronectin (FIG. 22).

This composition, obtained according to the method of the invention, before amplification of the mixture of the stromal vascular fraction and extracellular matrix, is a tissue composition. This composition further comprises mature adipocytes.

According to a fifth object, the invention relates to a kit comprising the composition as previously defined, the proliferation medium and the differentiation medium.

According to a sixth object, the invention relates to the in vitro use of the extracellular matrix as defined or the in vitro use of the composition as defined above for the screening and/or the characterisation of pharmacological active ingredients, in particular against obesity and/or associated metabolic diseases such as type 2 diabetes and cardiovascular diseases.

The invention further relates to brown or beige adipocytes obtained according to the method defined above, or derived from a composition as previously defined, and intended for use, or for the use thereof, in cell therapy, or for the treatment of metabolic disorders. The term “derived from” should be understood as meaning that the adipocytes originate from the composition, by differentiation of stem cells of brown or beige adipocytes.

The invention thus relates to a composition, obtained according to the methods of the invention, comprising brown or beige adipocytes or the precursors of such adipocytes, and intended for use, or for the use thereof, in cell therapy, or for the treatment of metabolic disorders. This composition is a tissue composition of human adipose tissue and comprises the entire product obtained by the methods of the invention, that is to say the mixture of the stromal vascular fraction and the extracellular matrix, after proliferation, or even after differentiation.

The brown or beige adipocytes or the precursors of such adipocytes, can be used for the treatment of obesity in overweight or obese individuals. To this end, and in one example, a sample is taken from a white adipose tissue in an individual. This taken tissue is then treated according to the method of the invention, in order to obtain, still in this example, products ExAdEx-tissue or even ExAdEx-lobules as previously described. These products, in which the precursor stem cells of brown or beige adipocytes have undergone an amplification, are then advantageously the subject of a differentiation into brown or beige adipocytes. Then, the products including these brown or beige adipocytes are transplanted, for example, into a white adipose tissue or in the vicinity of such a tissue of the individual. It should be noted that the products including the brown or beige adipocytes are a tissue composition as defined in the preceding paragraph. In another embodiment, brown or beige adipocytes are not transplanted, but stem cells of brown or beige adipocytes, having undergone the amplification according to the method of the invention, in other in other words a tissue composition as defined above, comprising the mixture of the stromal vascular fraction and the extracellular matrix after proliferation, and the differentiation into mature white or beiges adipocytes is performed in the body of the transplanted individual.

### EXAMPLES

### Example 1. Mechanical Extraction

### a) Mechanical Extraction Process for the Characterisation of the Cell and Matrix Populations During the Process

The mechanical extraction of the stromal vascular fraction and the extracellular matrix, from an adipose tissue sample from a human donor, can be carried out according to the following steps (FIGS. 1A and 1B)
1. Removal of adipose tissue by aspiration in a 10 cc sterile syringe equipped with a 2 mm Coleman cannula in −kPa negative pressure.
2. In order to separate the different phases, the syringe is centrifuged at 1600 rcf (relative centrifugal force), 3 min in the collection tube. The oil fraction as well as the blood fraction and anaesthetic liquid are eliminated. The pelleted fraction, named C1, is retained.
3. One unit of physiological saline is injected into the syringe, followed by an incubation for 30 min at 37° C. under stirring. The syringe is centrifuged at 1600 rcf, 3 min in the collection tube. The physiological fluid fraction and the oil fraction are eliminated. The pelleted fraction, named C2, is retained.
4. The syringe is connected to another male Luer-Lock type syringe connected by a Tulip® type connector in order to carry out the dissociation of the tissue by an emulsification. Three types of Tulip® connector, 2.4 mm, 1.4 mm then 1.2 mm, are successively used, over 30 passages.
5. One unit of physiological saline is injected into the syringe, followed by an incubation for 30 min at 37° C. under stirring. The syringe is centrifuged at 1600 rcf 3 min in the collection tube. The physiological fluid fraction and the oil fraction are eliminated. The pelleted fraction, named C3, is retained.
6. The contents of the syringe as well as the contents C1, C2 and C3 of the collection tubes which are previously cleared of blood cells are transferred through a sterile connection in a culture bag containing the EGM+ culture medium at 37° C. for the expansion phase.

During step 4 above of tissue dissociation, a connector of a brand other than the Tulip® brand can be used. The number of connectors used is comprised between 1 and 5. The number of passages through these connectors is comprised between 10 and 50.

FIG. 1A shows a method allowing gathering in step 2 the C1 and C2 populations as well as the matrices M1 and M2. In step 3 the population C3 and the matrices M3 and M4 are grouped.

### b) Characterisation of the Obtained Cell Populations

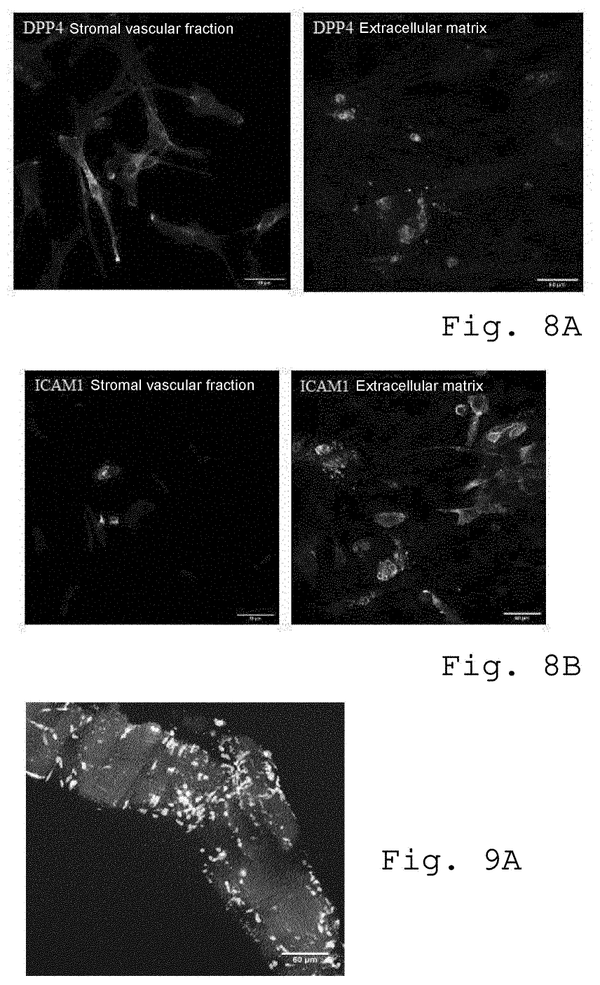

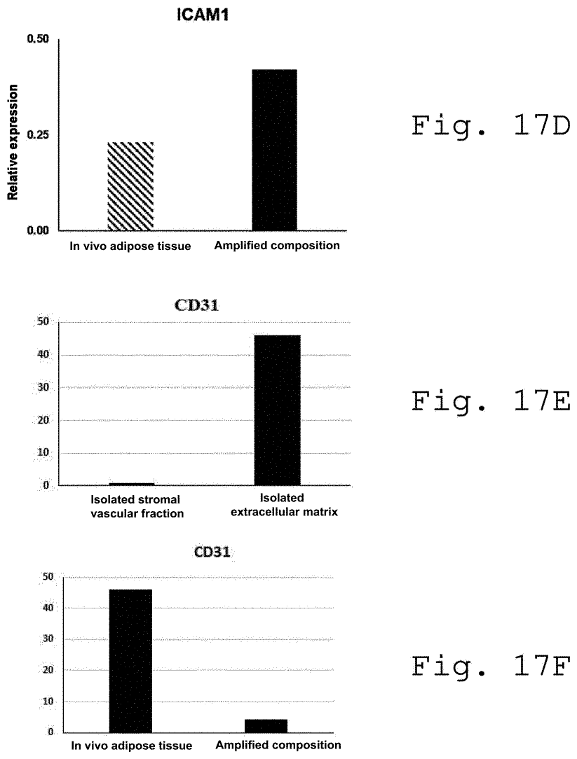

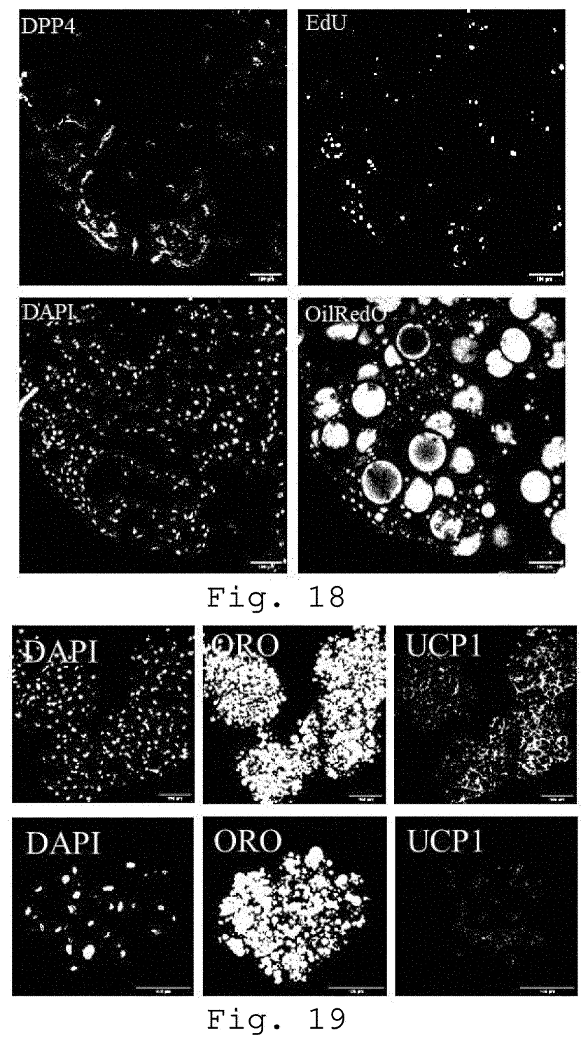

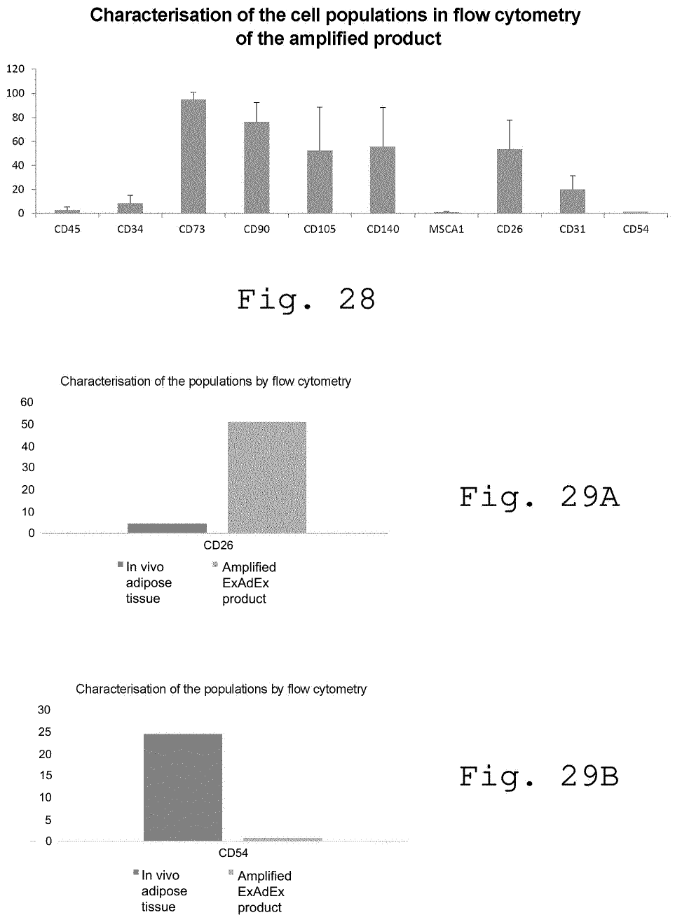

The process described above allows sequentially extracting the stromal vascular fraction and an extracellular matrix. The cell populations are characterised in particular by fluorescence microscopy, by a molecular characterisation by quantitative PCR and by flow cytometry.
The obtained stromal vascular fraction is characterised by a majority of DPP4+ type cells (FIG. 17A) at the level of gene expression of the marker DPP4 and by observation of the DPP4 protein by microscopy (FIG. 8A). In comparison, the ICAM1 protein is little present (FIG. 17C and FIG. 8B).
The obtained extracellular matrix is characterised by a majority of ICAM1+ type (FIG. 17C) and CD31+ cells (FIG. 17E) at the level of gene expression. This result is confirmed by the presence of cells expressing the ICAM1 protein observed by microscopy (FIG. 8B)
the obtained amplified ExAdEx product is characterised in particular by the presence of CD26* type cells (FIG. 28). In particular, profiles of the stem cells of brown or beige adipocytes of ex vivo adipose tissue and the amplified ExAdEx product, are obtained by determination, by flow cytometry, of the cell surface markers. It is thus demonstrated that the method of the invention allows an increase in the cell population expressing the marker CD26 from 4% to 51% (FIG. 29A). The cells expressing the marker CD54 are present, but not amplified by the method of the invention (FIG. 29B).

### c) Characterisation of the Matrices M1 to M4 Obtained During the Different Steps of the Method

The matrix obtained during step 2, named herein M1 is of fibrous type (FIG. 9C).
The matrix obtained during step 3, named herein M2 is of fibrous type and rich in collagen (FIG. 9D). The collagen is revealed by PicroSirius Red (FIG. 9A) which allows, in addition, visualising a rod-like collagen structure. This matrix contains endogenous cells.
The matrix obtained during step 5 and isolated in the collection tube, named herein M3, is of fibrous type (FIG. 9E and FIG. 9B). This matrix also contains endogenous cells. The collagen of the isolated matrix is revealed, in FIG. 9B, by the PicroSirius Red which stains the type I and type III collagen fibres. The obtained red colour (in shades of grey in FIG. 9B) indicates that the collagen remains organised, namely that the present collagen always has an a helical secondary structure and a triple helix quaternary structure. It is not degraded. Indeed, disorganised collagen is stained green by PicroSirius Red.
The matrix obtained during step 5 and contained in the syringe, named herein M4, is composed of a majority of mature adipocytes and a type I collagen framework (FIG. 10B). The matrix M4 is also composed of capillary structures formed by CD31+ endothelial cells (FIG. 10C) and by a network of PDGFRa+ stem cells of adipose tissue (FIG. 10D).

### d) Purification of the C1/C2 Infratant

The populations C1 and C2 are previously cleared of blood cells by incubating the cell pellets in a 1×lysis buffer comprising ammonium chloride, called ACL, diluted in sterilised water, in a buffer: sample ratio, ranging from 1:1 to 1:10, at a temperature comprised between 4 and 37° C. and during an incubation time varying from 5 minutes to 30 minutes. In order to determine the effect of this ACL treatment on the stem cells of brown or beige adipocytes of the adipose tissue, a flow cytometry analysis was carried out. Thus, a total of 1.10⁵ stem cells of human adipose tissue were incubated in a saline solution as a control, or at different times, in 10 volumes of the blood cell lysis solution. FIG. 25A illustrates the direct viability in terms of numbers of viable cells after incubation in the different compositions. FIG. 25B illustrates the viability after 24 hours of culture and FIG. 25C has the proliferation capacity after 5 days under adherent culture conditions (Tukey's post-hoc test *<0.05; **<0.01; ***<0.001 for n=5). A graphic result of this analysis by flow cytometry is shown in FIGS. 26A, 26B, 26C and 26D and allows visualising the number of viable and dead cells after different treatment times in the ACL. A saline solution is taken as a reference. In these figures, in the grey box on the left, the viable cells appear and, in the box on the right, the dead cells appear, identified by Propidium Iodide marking. This marking has the property of only penetrating the cells whose cell membrane is damaged. Another analysis by flow cytometry was also carried out in order to determine the influence of the purification of the infranatant on endothelial cells of adipose tissue. Thus, a total of 1.10⁵ endothelial cells of human adipose tissue were incubated in a saline solution as a control, or at different times, in 10 volumes of the blood cell lysis solution. FIG. 27A illustrates the direct viability in terms of numbers of viable cells after incubation in the different compositions. FIG. 27B illustrates the viability after 24 h of culture and FIG. 27C shows the proliferation capacity after 5 days under adherent culture conditions. It results from these analyses that an incubation period in the ACL which is greater than 5 minutes damages the cell viability and the proliferation capacities of the stem cells of adipose tissue contained in the infranatant. The incubation in the ACL has, moreover, no effects on the viability or the proliferation capacities of the endothelial cells of human adipose tissue.

### Example 2. Cell Expansion and Differentiation

A method for ex vivo expansion of stem cells of brown or beige adipocytes of adipose tissue and differentiation in an adipose tissue-mimicking environment comprises the following steps:
1. The final product obtained in Example 1 containing the populations C1-C3 as well as the matrices called EndoStem Matrix M1-M4 are cultured in suspension in bags and maintained in the EGM+ proliferation medium with a stirring for 24 hours at 37° C. 5% CO2, then maintained under the same conditions, preferably under stirring.
2. The EGM+ proliferation medium is changed at 50% every two days.
3. A mechanical dissociation in a closed system, by passage through 2 syringes or two culture bags mounted in tulip, is performed on day 5 and day 10.
4. On day 14, EGM+ proliferation medium is replaced by the differentiation cocktail I composed of EGM+ enriched with 250 μM Dexamethasone; 500 μM IBMX; 1 μM Rosiglitazone; 2 μM T3 and 2.5 μg/ml insulin.
5. On day 17, the differentiation medium I is replaced by the differentiation medium II composed of EGM+ enriched with 1 μM Rosiglitazone; 2 μM T3 and 2.5 μg/ml insulin.

### Example 3. Characterisation of the Amplification Capacity of the Matrix

The extracellular matrices called EndoStem-Matrix of the invention have been characterised, in particular, by fluorescence microscopy, in the presence of different specific markers. Proliferating cells have thus been detected by incorporation, during the phase of DNA replication, of fluorescent Edu (5-ethylnyl-2′-deoxyuridine) into the matrices EndoStem-Matrix of the invention, as illustrated in FIG. 11, proving that these are bioactive. Indeed, FIG. 11 shows that the cells which are endogenous to the matrices are maintained in proliferation during the amplification phase.

Moreover, FIG. 12 highlights the presence of exogenous stem cells of adipose tissue co-cultured after three days of co-culture with the extracellular matrix of the invention. The extracellular matrix therefore allows providing a support for the proliferation of the stromal vascular fraction: the added stem cells of adipose tissue may be attached to the matrix EndoStem-Matrix, in suspension. This shows that exogenous cells have the ability to be attached to the extracellular matrix according to the invention, and demonstrates that the matrix can be used as such and alone for clinical or in vitro applications and, in particular, for the screening and/or the characterisation of pharmacological active ingredients. The exogenous stem cells can be are genetically modified, for example, to express a protein of interest. The exogenous stem cells can moreover be non-adipocyte or specifically adipocyte stem cells. It can be, for example, skin stem cells, in particular epithelial stem cells of the skin or induced pluripotent stem cells.

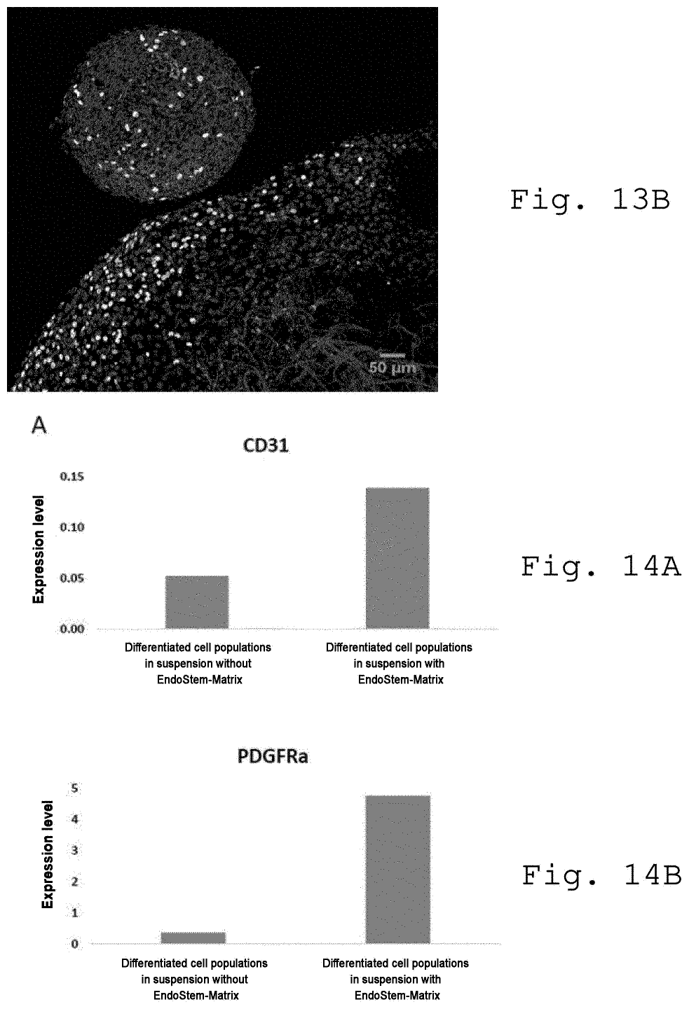

With reference to FIG. 13B, the stromal vascular fraction is amplified by its culture on the EndoStem Matrix of the invention. Conversely, with reference to FIG. 13A, when the stromal vascular fraction is cultured in suspension without the extracellular matrix, cell aggregates without proliferation are observed. The extracellular matrix of the invention therefore has the ability to amplify the added stem cells of adipose tissue.

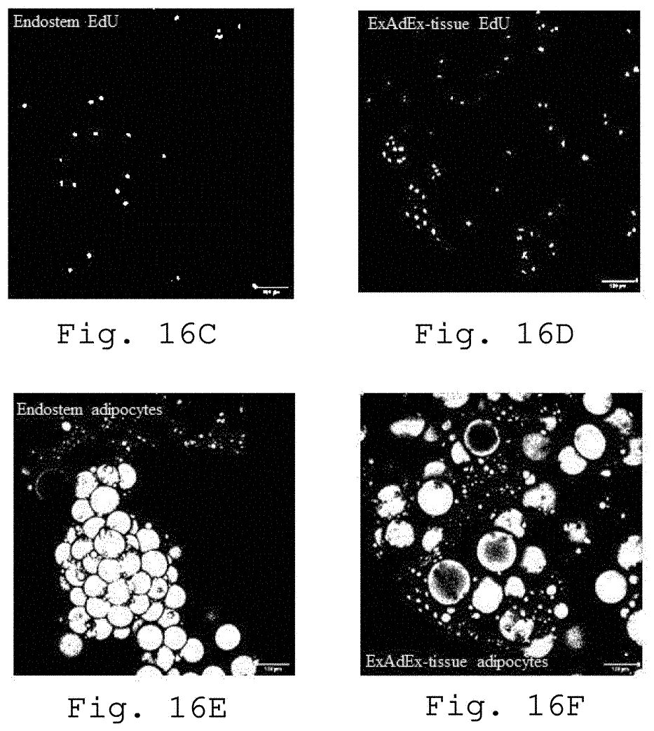

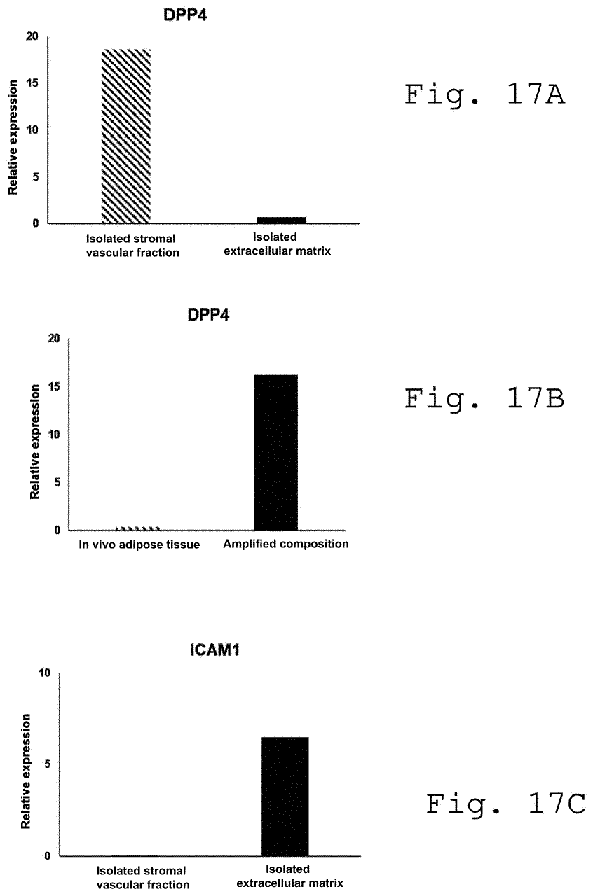

In addition, FIG. 16 allows highlighting the need for co-culture of the stromal vascular fraction and the matrix to obtain an amplification of the stem cells of adipose tissue. FIGS. 16A, 16C and 16E show a low proportion of proliferating cells in the cultured Endostem matrix without the addition of the stromal vascular fraction. In comparison, FIGS. 16B, 16D and 16F show that the co-culture of the stromal vascular fraction and the extracellular matrix allows obtaining a superior proliferation of stem cells of adipose tissue. Similarly, in order to compare the viability and the amplification capabilities of the stem cell of adipose tissue, cells contained in a composition without adding the stromal vascular fraction (FIG. 32A) or a composition comprising a mixture of the extracellular matrix and cells isolated from the infranatant (FIG. 32B) are isolated by mechanical digestion, cultured under adherent conditions for 48 hours, then fixed and stained with violet crystal, thus allowing observing the cell density. The cells contained in the cell well are shown in black in FIG. 32. FIG. 32A thus shows a weak amplification of the cell population in the absence of the addition of the stromal vascular fraction. Conversely, FIG. 32B shows an amplification of the cell populations of the stromal vascular fraction after culturing.

The cell amplification capacity of the different matrices M1 to M4 obtained during the steps of the example 1 has been verified. Thus, about 104 stem cells of adipose tissue were maintained in suspension in the presence of the different matrices M1 to M4 in Ultra Low Attachment (ULA) wells. Eight days later, the cells are detached from the matrix by trypsin/EDTA then counted. The obtained values are shown in the following table 1:

TABLE 1

| | Number | Amplification |
|---|---|---|
| Conditions | of cells | factor |
| Stem cells of | 2.10⁴ | 1 |
| adipose tissue | | |
| without matrix | | |
| Stem cells of | 5.10⁴ | 2.5 |
| adipose tissue | | |
| with matrix M1 | | |
| Stem cells of | 53.10⁴ | 26.5 |
| adipose tissue | | |
| with matrix M2 | | |
| Stem cells of | 7.4.10⁴ | 3.7 |
| adipose tissue | | |
| with matrix M3 | | |
| Stem cells of | 72.10⁴ | 36 |
| adipose tissue | | |
| with matrix M4 | | |
| Matrix M4 without | 5.10⁴ | — |
| the addition of | | |
| stem cells of | | |
| adipose tissue | | |

In the table above, for the particular case of the individual matrices M1 to M4, the amplification factor is the ratio between the number of cells obtained after culture in the presence of the extracellular matrix and the number of cells obtained in the absence of the extracellular matrix.

The matrices M2 and M4 have a strong amplifying power of the stem cells of adipose tissue. The obtained matrix volume M2 is very small compared to the volume of the M4 (FIG. 10A). The matrix M4 illustrates an extracellular matrix as defined in the invention.

The level of expression of different cellular markers (marker of stem cells of adipose tissue and endothelial cells CD31) was analysed after culture in suspension of the stromal vascular fraction on the extracellular matrix of the invention in the proliferation medium. This study reveals an amplification of the DPP4 stem cells of brown/beige adipose tissue (FIG. 17B), a conservation of ICAM1 precursor stem cells of adipocytes of human adipose tissue (FIG. 17D) and preservation of an endothelial cell population (FIG. 17F).

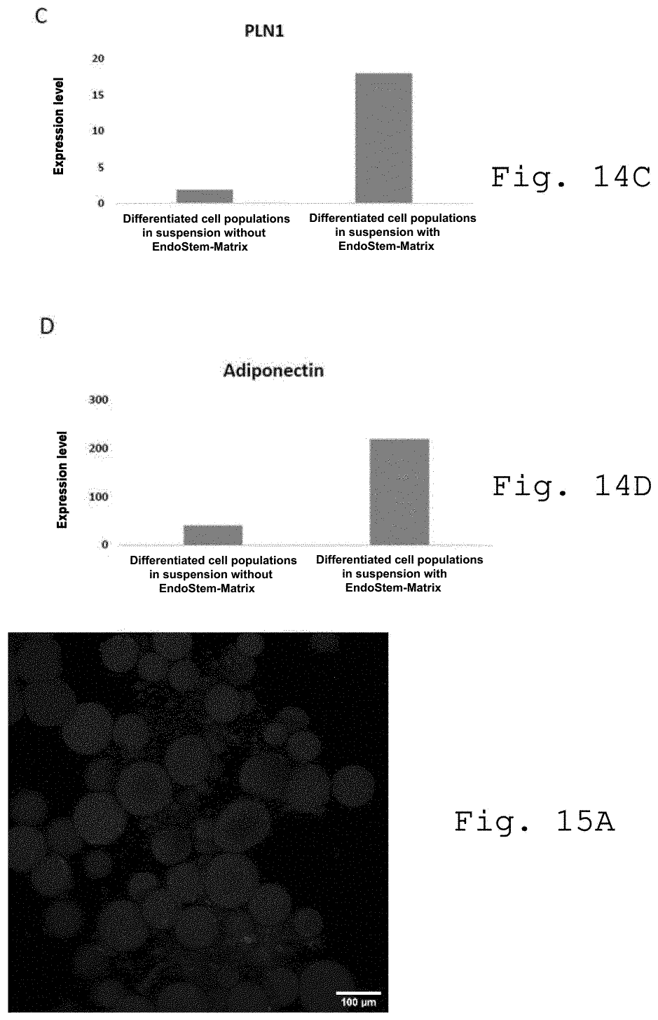

The level of expression of different cellular markers (endothelial cell marker CD31, adipocyte stem cell marker PDGFRa, and two mature adipocyte markers PLN1 and Adiponectin) was analysed after culture in suspension of the stromal vascular fraction on the extracellular matrix of the invention after the step of amplification and differentiation of the product ExAdEx-tissue. FIG. 14 shows a comparison of these levels of expression with those resulting from a culture in suspension of the stromal vascular fraction without the extracellular matrix of the invention. This study reveals an amplification of the endothelial cells of the vascular network of adipose tissue (FIG. 14A) and the stem cells of adipose tissue (FIG. 14B). This study also allows highlighting the best differentiation ability induced by the extracellular matrix of the invention (FIGS. 14C and 14D). Thus, the 3D amplified stem cells of adipose tissue on the extracellular matrix of the invention retain their ability to be differentiated into adipocytes.

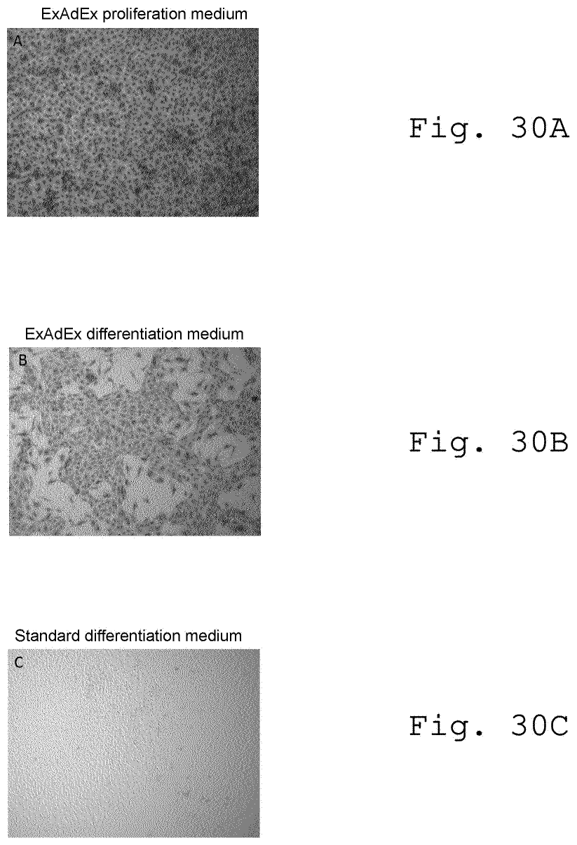

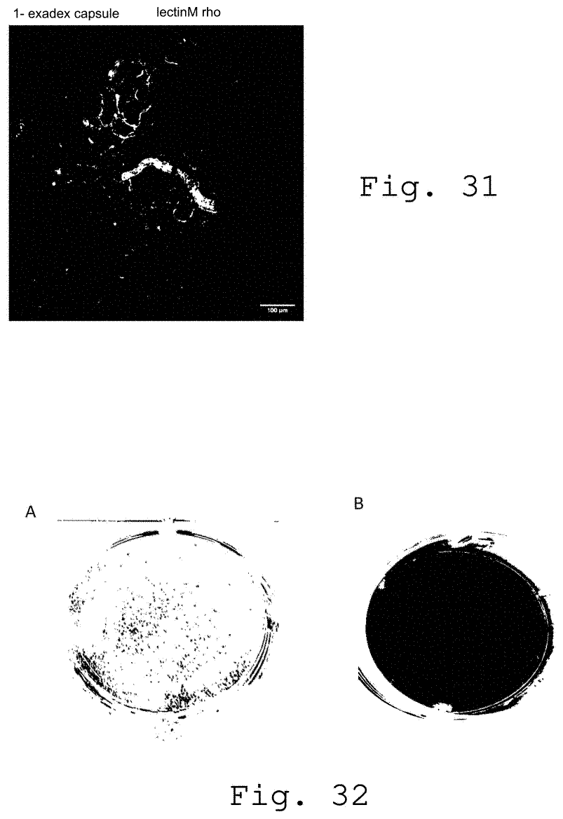

The ExAdEx process of the invention also allows preserving the native vascular network, during the cell amplification and differentiation. FIGS. 30A, 30B and 30C illustrate the endothelial cells isolated from human adipose tissue after culture in different media. FIG. 30A shows the endothelial cells of adipose tissue after culture in the proliferation medium of the invention. FIG. 30B shows the endothelial cells of adipose tissue after culture in the differentiation medium of the invention.

And FIG. 30C shows the endothelial cells of adipose tissue after culture in a standard, non-optimised, differentiation medium which comprises DMEM and serum. As a result, only the media which are described in the invention allow preserving the endothelial cells during the steps of proliferation and differentiation into brown/beige adipose tissue. The preservation of endothelial cells is of interest, in particular, for transplantation applications of the brown/beige adipose tissue, allowing a post-transplant vascularisation and therefore a viability of the transplantation product. In this regard, it is also demonstrated herein that the native vascular network, present in the amplified composition, and possibly differentiated, obtained by the method of the invention, has post-transplant revascularisation capacities in Nude mice. Indeed, as it appears in white in FIG. 31, 4 weeks after transplantation in Nude mice, the composition of the invention has a functional vascular network.

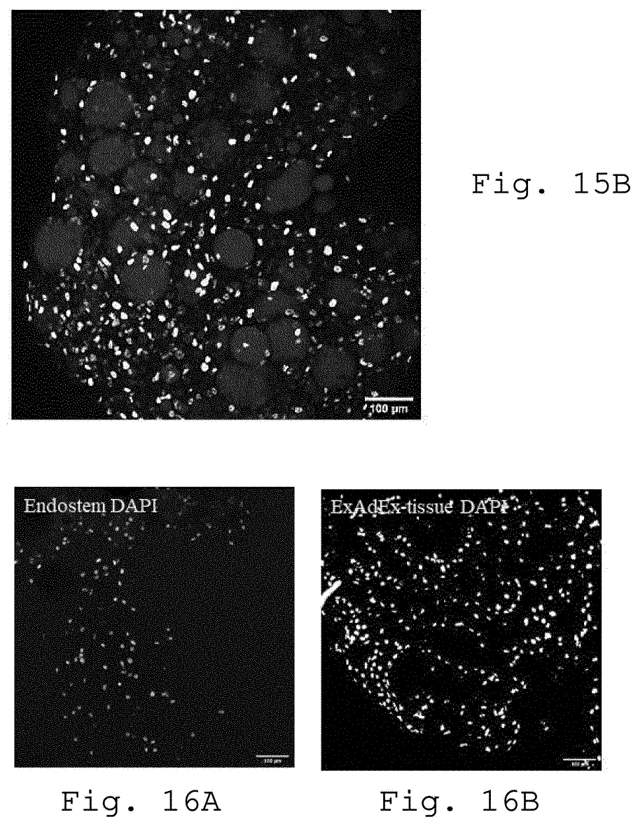

It will be noted that an undissociated adipose tissue, which can be assimilated to an explant, remains viable for a short time ex vivo. Thus, as shown in particular in FIG. 15, the matrix which is isolated by dissociation contains proliferating cells (FIG. 15B), unlike an undissociated tissue (FIG. 15A). FIGS. 15A and 15B allow comparing the cell proliferation in the undissociated tissue (FIG. 15A) and in the isolated matrix (FIG. 15B). In FIG. 15A, the undissociated adipose tissue does not shows proliferating cells. In FIG. 15B, the composition shows proliferating cells. Indeed, this figure shows, in white, the nuclei of the proliferating cells.

It will be noted that the cells which are isolated according to the invention, by centrifugation of the washing liquid, are molecularly characterised by the marker DPP4. DPP4 is a marker of the precursor cells of the ICAM1 pre-adipocytes, which have a great proliferation capacity and which are localised in the interstitial reticulum of adipose tissue. These are cells which have the ability to proliferate in the composition according to the invention. It is important to note that these cells are eliminated following washing carried out according to the methods of the prior art. As well as this is shown in FIG. 17A, the expression of DPP4 is concentrated in the isolated stromal vascular fraction. The matrix expresses little. However, and as shown in FIG. 17C, the expression of ICAM1, is concentrated in the isolated matrix, as well as the CD31 type cells in FIG. 17E. The cells which carry the amplification in the composition are the added cells expressing DPP4.

Indeed, FIG. 18 shows that in the co-culture product, the cells expressing the Edu proliferation marker are the cells also expressing the marker DPP4. Thus, the stem cells, which carry the amplification power, are preferentially the DPP4 or CD26+ type cells.

Moreover, it should be noted that in vivo adipose tissue contains macrophages and that the amplified composition according to the invention maintains the presence of macrophages of type M1, as shown in FIG. 21A, and of type M2, as shown in FIG. 21B. in FIG. 21A, M1 type macrophages are revealed by the marker IL-1b and, in FIG. 21B, the M2 type macrophages are revealed by the marker MRCI1.

Finally, and as shown in FIG. 22, the isolated matrix according to the invention comprises proteins of the extracellular matrix, namely in particular, the type I collagen, the type IV collagen, elastin, fibronectin, laminin. The CD31 endothelial cell marking shows that a capillary network is preserved.

### Example 4 Amplification/Differentiation into Browns/Beiges Adipocytes

FIG. 19 shows that it is the positive DPP4 cells which are preferentially the precursors of brown/beige adipocytes. Indeed, an isolated population of stem cells of human adipose tissue expressing DPP4+ and differentiated according to the medium described in the invention (FIG. 19 top line), shows an expression of the marker UCP1 in confocal microscopy. However, a stem cell population of human adipose tissue not expressing the surface marker DPF4, does not show an expression of the marker UCP1 after differentiation. Molecular analyses confirm these observations and show that the adipocyte differentiation marker PLIN1 is comparable for the positive and negative DPP4 populations (FIG. 20A), however, the marker of brown/beige adipose tissue differentiation is observable only in the population which is differentiated from stem cells expressing the marker DPP4 (FIG. 20B).

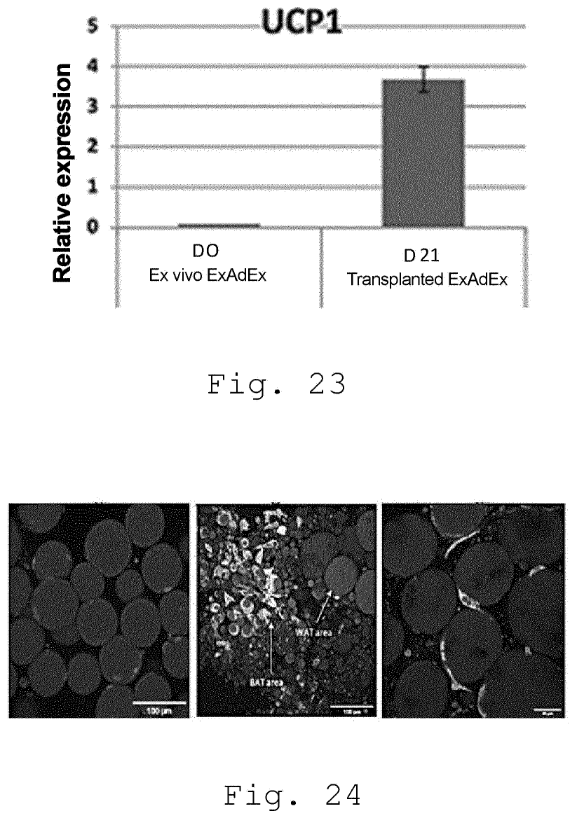

The amplified product ExAdEx-tissue—but not differentiated—was injected at the interscapular level, in the vicinity of the brown adipose tissue of immunodeficient mouse called “Nude”. Twenty-one days after injection (D21) the product ExAdEx-tissue was sampled. RNAs were extracted from constituent cells of the sampled tissue. The expression level of the brown/beige adipocyte marker UCP1 was compared to that of the product before injection (J0). The UCP1 expression levels at D0 and D21 were determined by real-time quantitative PCR then normalised by relating them to a reference gene whose expression does not vary under the 2 conditions, namely the gene: human GUSB (beta glucuronidase). As shown in FIG. 23, it appears that the amplified transplanted tissue was subjected to an in vivo cell differentiation into brown/beige adipocytes after transplantation. In practice, and as shown in FIG. 24, there are no brown/beige adipocytes in the white subcutaneous adipose tissue before implementation of the method of the invention (FIG. 24, photograph on the left). The presence of brown/beige adipocytes (specifically marked by expression of the UCP1 protein) ex vivo after the invention from adipose tissue of overweight person (FIG. 24, photograph of the centre). The presence of brown/beige adipocytes ex vivo after the invention from adipose tissue of a patient with severe obesity (FIG. 24, photograph on the right).

1. A method for in vitro or ex vivo obtaining brown or beige adipocytes from adipose tissue, the method comprising:
extracting (i) a stromal vascular fraction from a human adipose tissue comprising endothelial cells of a vascular network of human adipose tissue and stem cells of human adipose tissue and (ii) an extracellular matrix of the human adipose tissue, the extracellular matrix comprising endothelial cells of the vascular network of human adipose tissue, stem cells of human adipose tissue and collagen, the extracting of the extracellular matrix comprising a mechanical dissociation;
mixing the stromal vascular fraction and the extracellular matrix;
culturing the mixture obtained in the mixing, in suspension, in a cell proliferation medium of the stem cells of brown or beige adipocytes of adipose tissue; and
inducing a differentiation of the stem cells of brown or beige adipocytes of adipose tissue to obtain brown or beige adipocytes;
wherein the extracting of the stromal vascular fraction and the extracellular matrix comprises:
performing centrifugation of human adipose tissue to obtain at least two distinct fractions, a fraction A comprising a centrifuged extracellular matrix, and the stromal vascular fraction; and
performing mechanical dissociation of the fraction A to obtain the extracellular matrix.

2. The method according to claim 1, wherein the differentiation of the stem cells of adipose tissue is induced in a differentiation medium.

3. The method according to claim 1, wherein the mechanical dissociation is a dissociation which does not involve collagenase.

4. The method according to claim 3, wherein the mechanical dissociation is a non-enzymatic dissociation.

5. The method according to claim 1, wherein the method further includes eliminating blood cells present in the stromal vascular fraction.

6. The method according to claim 1, wherein the collagen of the extracellular matrix is structured collagen, which has a fibrillar organisation.

7. The method according to claim 1, wherein the culturing of the mixture of the stromal vascular fraction and the extracellular matrix comprises:
transferring the mixture, in a sterile manner, into a suspension culture bag comprising the proliferation medium; and
amplifying the mixture to obtain an amplified mixture comprising cell clusters.

8. The method according to claim 7, wherein the amplified mixture comprising the cell clusters is subjected to a mechanical dissociation to obtain cell aggregates.

9. The method according to claim 1, wherein the method comprises adding, in the proliferation medium, a serum, a fibroblast growth factor and an insulin-like growth factor.

10. The method according to claim 2, wherein the method comprises adding, in the differentiation medium, Rosiglitazone and/or SB431542.

11. The method according to claim 1, wherein the method further includes performing, sell sorting aimed at sorting stem cells expressing surface marker DPP4.

12. An extracellular matrix extracted according to the method according to claim 1, the extracellular matrix comprising endothelial cells of the vascular network of human adipose tissue, stem cells of brown or beige adipocytes of human adipose tissue, and collagen.

13. A composition comprising a mixture of the extracellular matrix according to claim 13 and of a stromal vascular fraction extracted according to the method according to claim 1.

14. A kit comprising the composition according to claim 13, a cell proliferation medium comprising serum, a fibroblast growth factor and an insulin-like factor, and a differentiation medium comprising rosiglitazone and/or SB431542.

15. A method of screening and/or characterizing pharmaceutical active ingredients, comprising performing the screening and/or characterizing using the extracellular matrix according to claim 12.

16. A method of screening and/or characterizing pharmaceutical active ingredients, comprising performing the screening and/or characterizing using the composition according to claim 13.

17. A method of screening and/or characterizing pharmaceutical active ingredients, comprising performing the screening and/or characterizing using the kit according to claim 14.

18. A pharmaceutical product adapted for cell therapy of treatment of metabolic disorders, comprising brown or beige adipocytes obtained by the method according to claim 1.

19. A pharmaceutical product adapted for treatment of obesity comprising brown or beige adipocytes obtained by the method according to claim 1.

20. A differentiation medium adapted for differentiating stem cells of brown or beige adipocytes into brown or beige adipocytes according to the method according to claim 1, comprising an endothelial cell growth medium supplemented with serum, growth factors, rosiglitazone and SB431542.
